(19) United States
(12) Patent Application Publication
(10) Pub. No.: US 2012/0157385 A1
(43) Pub. Date: Jun. 21, 2012

(54) CYCLOSPORIN CONJUGATES

(75) Inventors: David Selwood, London (GB); Martin Crompton, London (GB)

(73) Assignee: UCL Business PLC, London (GB)

(21) Appl. No.: 13/386,317

(22) PCT Filed: Jul. 19, 2010
(86) PCT No.: PCT/GB2010/001369

§ 371 Date: Mar. 2, 2012

(30) Foreign Application Priority Data
Jul. 20, 2009 (GB) 0912584.0

(51) Int. Cl. A61K 38/13; C12Q 1/02; A61P 9/10; G01N 33/53; C07K 7/64; A61P 25/28

(52) U.S. Cl. 514/15.1; 530/321; 514/20.5; 514/17.7; 435/7.8; 435/29

(57) ABSTRACT

A conjugate which comprises a cyclosporin moiety of formula (I) linked to one or more mitochondrial targeting groups, or a pharmaceutically acceptable salt thereof: wherein: A represents or, B represents methyl or ethyl, one Of R₁ and R₁ represents hydrogen and the other represents methyl, R₂ represents ethyl or isopropyl, R³ represents hydrogen or methyl, and R⁴ represents —CH₂CH(CH₃)CH₃, —CH₂CH(CH₃)CH₂CH₃, —CH(CH₃)CH₃ or —CH(CH₃)CH₂CH₃.

### BACKGROUND

Ischaemic diseases, notably myocardial infarction and stroke, are the leading cause of death and disability throughout the world. Following an ischaemic episode, early restoration of blood flow is essential to restrict tissue damage. However, when blood supply is restored to ischaemic cells, the newly returning blood can adversely affect the damaged tissue. This is known as reperfusion injury, and often causes further damage and cell death following an ischaemic episode. It is therefore a therapeutic goal to mitigate and avoid ischaemia/reperfusion (I/R) injury. There are currently no effective therapeutic treatments for ischaemia/reperfusion injury.

Cyclosporin A (CsA) is well known as an immunosuppressive drug. It has been proposed for use in treating ischaemia/reperfusion injury (see N. Engl. J. Med. 395; 5 473 to 481). However, experimental models and pilot trials to investigate the efficacy of cyclosporin in treating ischaemia/reperfusion have yielded highly variable and only marginal effects.

It is a finding of the present invention that ischaemia/reperfusion injury can be treated by selective inhibition of mitochondrial cyclophilin D (CyP-D). It has also been found that simultaneous inhibition of cytosolic cyclophilins, such as cyclophilin A (CyP-A), partially or completely offset the beneficial effects of cyclophilin D inhibition.

Mitochondrial cyclophilin D (hereinafter “cyclophilin D”) is a peptidylprolyl cis-trans-isomerase in the cyclophilin family. It is also known as cyclophilin F and peptidylprolyl isomerase F. Cyclophilin D is located in the mitochondrial matrix. Cyclophilin inhibitors which are designed to accumulate in the mitochondria will therefore have some selectivity for cyclophilin D.

### SUMMARY OF THE INVENTION

The present invention therefore provides a conjugate which comprises a cyclosporin moiety of formula (I) linked to one or more mitochondrial targeting groups, or a pharmaceutically acceptable salt thereof:

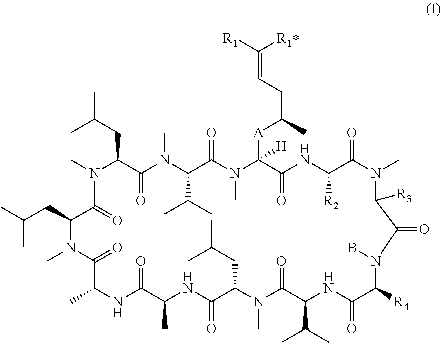

wherein:
A represents

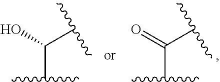

B represents methyl or ethyl, one of R₁ and R₁* represents hydrogen and the other represents methyl,
R₂ represents ethyl or isopropyl,
R₃ represents hydrogen or methyl, and
R₄ represents —CH₂CH(CH₃)CH₃, —CH₂CH(CH₃)CH₂CH₃, —CH(CH₃)CH₃ or —CH(CH₃)CH₂CH₃.

### DESCRIPTION OF THE FIGURES

FIG. 1 is a graph showing inhibition of isolated cyclophilin D by cyclosporin and a conjugate of the invention (Compound 1).

FIG. 2 is a graph showing that complexes of Compound 1 and cyclophilin A do not inhibit calcineurin.

FIG. 3 is a series of graphs showing that Compound 1 preferentially inhibits intramitochondrial cyclophilin D rather than extramitochondrial cyclophilin A.

FIG. 4 is a series of graphs and diagrams showing that Compound 1 preferentially inhibits intramitochondrial cyclophilin D rather than extramitochondrial cyclophilin A in B50 neuronal cells.

FIG. 5 shows that Compound 1 is a better cytoprotectant than cyclosporin in hippocampal neurons following transient deprivation of glucose and oxygen.

FIG. 6 is a graph showing cytoprotection by a series of conjugates of the invention (Compounds 1 to 4) against pseudo ischaemia/reperfusion induced necrosis in rat hippocampal neurons.

FIG. 7 is a graph showing that deprivation of oxygen and glucose for 4 hours induced negligible necrosis in rat heart cells.

FIG. 8 is a graph showing that reoxygenation of rat heart cells following oxygen and glucose deprivation induces progressive cell death of the heart cells. That cell death is inhibited by Compound 2.

FIG. 9 is a graph comparing the cytoprotective properties of Compounds 2 and 3 with those of CsA in rat heart cells.

### DETAILED DESCRIPTION OF THE INVENTION

Typically, the cyclosporin moiety of formula (I) is linked to one, two, three or four mitochondrial targeting groups. Preferably, said cyclosporin moiety is linked to one or two mitochondrial targeting groups, more preferably to one mitochondrial targeting group.

When said cyclosporin moiety is attached to more than one mitochondrial targeting group, each mitochondrial targeting group can be the same or different.

Preferably in the cyclosporin moiety of formula (I):

A represents

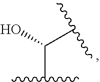

B represents methyl, R₁ represents methyl, R₁* represents hydrogen, R₂ represents ethyl, R₃ represents hydrogen, and R₄ represents —CH₂CH(CH₃)CH₃. That compound is cyclosporin A. It has the following formula:

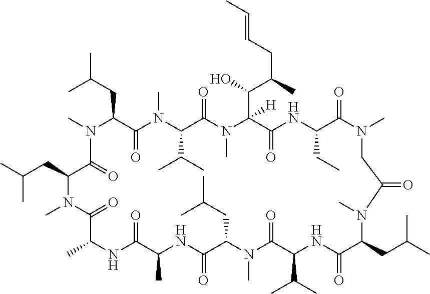

The residue at the 1 position of the cyclosporin moiety of formula (I) contains either a hydroxyl group or a ketone, depending on the identity of A. Thus, the residue at the 1 position is of formula (X) if A represents

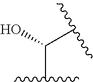

and of formula (X′) if A represents

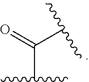

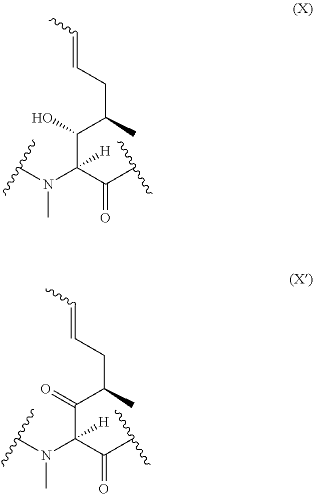

Typically, the or each mitochondrial targeting group is linked to the cyclosporin moiety covalently or non-covalently. Preferably all of the mitochondrial targeting groups are linked covalently or all of the mitochondrial targeting groups are linked non-covalently.

Preferably at least one of the mitochondrial targeting groups is linked covalently. More preferably all of the mitochondrial targeting groups are linked covalently.

The or each mitochondrial targeting group can be linked to the cyclosporin moiety directly or via a linker (L).

Preferably all of the mitochondrial targeting groups are linked directly to the cyclosporin moiety or all of the mitochondrial targeting groups are linked via a linker to the cyclosporin moiety.

Preferably at least one mitochondrial targeting group is linked via a linker to the cyclosporin. More preferably all of the mitochondrial targeting groups are linked to the cyclosporin moiety via linkers.

The nature of the linker (L) is not an important part of the invention. Thus, L can be any moiety capable of linking said mitochondrial targeting group to said cyclosporin moiety. Such linker moieties are well known in the art.

Typically the linker (L) has a molecular weight of 50 to 1000, preferably 100 to 500.

Typically the linker (L) is a straight chain C₁ to C₂₀ alkylene which is unsubstituted or substituted by one or more substituents selected from halogen atoms, hydroxy, alkoxy, alkyl, hydroxyalkyl, haloalkyl and haloalkoxy substituents, wherein zero or one to ten, preferably one to five, carbon atoms in the alkylene chain are replaced by spacer moieties selected from arylene, —O—, —S—, —NR′—, —C(O)NR′— and —C(O)— moieties, wherein R′ is hydrogen or C₁ to C₆ alkyl, preferably hydrogen, and the arylene moiety is unsubstituted or substituted by one, two or three substituents selected from halogen atoms, hydroxy, alkyl and alkoxy groups.

Typically said spacer moieties are selected from arylene, —O—, —S—, —NR′— and —C(O)NR′— moieties. Preferably said spacer moieties comprise 0 to 2 arylene, 0 to 2 -S—, 0 to 2 -O—, 0 to 2 -NR′— and 1 to 2 -C(O)NR′— moieties.

More preferably said spacer moieties comprise 0 to 2 arylene, 0 to 1 -O—, 0 to 1 -NH— and 1 to 2 -C(O)NH— moieties, for example (a) 1 arylene and 2 -C(O)NH— moieties, (b) 2 —C(O)NH— and 1 -O— moieties, (c) 1 arylene, 2 -C(O)NH— and 1 -O— moieties, or (d) 1 arylene, 1 -C(O)NH— and 1 -NH— moieties.

Preferably, said straight chain C₁-C₂₀ alkylene is unsubstituted or substituted by one or more, preferably 1 or 2, halogen atoms. Most preferably, said alkylene group is unsubstituted.

Preferably, the arylene spacer moiety is unsubstituted or substituted with one, two or three halogen atoms or hydroxy groups. When the arylene spacer moiety carries 2 or more substituents, the substituents may be the same or different. Most preferably the arylene spacer moiety is unsubstituted.

A mitochondrial targeting group is a group which is capable of concentrating the conjugate in the mitochondria of a cell. Thus, following incubation of a cell with a conjugate comprising one or more mitochondrial targeting groups, the concentration of the conjugate in the mitochondria will be higher than the concentration of conjugate in the cytosol.

Preferably, 15 minutes after application of the conjugate to the cell, the ratio of the concentration of the conjugate in the mitochondria to the concentration of the conjugate in the cytosol is greater than 1.5:1, more preferably greater than 2:1, more preferably greater 5:1, most preferably greater 10:1.

The specific structure of the mitochondrial targeting group in the conjugates of the invention is not vital. Mitochondrial targeting groups are well known. They have previously been used for directing, for example, antioxidant compounds to the mitochondria.

Examples of appropriate mitochondrial targeting groups are discussed extensively in the literature:
Souza et al, Mitochondrion 5 (2005) 352-358;
Kang et al, The Journal of Clinical Investigation, 119, 3, 454-464;
Horton et al, Chemistry and Biology 15, 375-382;
Wang et al, J. Med. Chem., 2007, 50 (21), 5057-5069;
Souza et al, Journal of Controlled Release 92 (2003) 189-197;
Maiti et al, Angew. Chem. Int. Ed. 2007, 46, 5880-5884;
Kanai et al, Org. Biomol. Chem. 2007, 5, 307-309;
Senkal et al, J Pharmacol Exp Ther. 317(3), 1188-1199;
Weiss et al, Proc Natl Acad Sci USA, 84, 5444-5488;
Zimmer G, et al. Br J. Pharmacol. 1998, 123(6), 1154-8;
Modica-Napolitano et al, Cancer Res. 1996, 56, 544-550;
Murphy et al (2007), Ann Rev. Pharm ToxiCol. 47, 629-656; and
Hoye et al, Accounts of Chemical Research, 41, 1, 87-97.

All of the above documents are incorporated by reference. For the avoidance of doubt, all of the mitochondrial targeting groups disclosed in these articles can be used in the conjugates of the present invention.

Typically, the mitochondrial targeting groups are those which have a Pearson's correlation coefficient (Rr) of greater than 0.1, preferably greater 0.2, more preferably greater than 0.4, for example 0.5 to 0.6, as determined by an assay which comprises the following steps:

(a) removing commercially available HeLa cells from a culture medium and washing the cells with phosphate-buffered saline;
(b) conjugating the mitochondrial targeting group to the commerically available fluorophore, to;
(c) incubating the cells from step (a) in 5 μM of the conjugate obtained from step (b) in serum-free minimum essential medium for 90 minutes;
(d) adding a reagent capable of labelling the mitochondria of the cells; and
(e) analysing fluorescence images of the cells to determine Pearson's correlation coefficient (Rr).

The above assay is described in more detail in Horton et al, Chemistry and Biology 15, 375-382.

Typically, the pH of the phosphate-buffered saline in step (a) is pH 7.4.

Typically, the reagent in step (d) is Mitotracker CMXRos, which is commercially available from Invitrogen. Typically, Mitotracker CMXRos is added at a concentration of 50 nM for the last 15 minutes of the incubation in step (c).

Typically, following step (d), the cells are washed three times with serum-free minimum essential medium and placed on ice.

Typically, fluorescence images are taken of the cells in step (e) with an inverted Zeiss LSM 510 confocal microscope and analyzed with Colocalizer Pro software to calculate Pearson's correlation coefficient (Rr).

Particularly preferred mitochondrial targeting groups are groups which are capable of concentrating the conjugate specifically in the mitochondrial matrix of a cell. Thus, a conjugate of the invention preferably has a mitochondrial matrix/extramitochondrial accumulation ratio of greater than 2, more preferably greater than 3, more preferably greater than 4, as determined by an assay which comprises the following steps:
(1) preparation of a first suspension of isolated mitochondria and recombinant cyclophilin A in buffer solution;
(2) addition of the conjugate to the suspension obtained in (1);
(3) addition of Ca²⁺ to the suspension obtained in (2) to a concentration of 50 μM;
(4) monitoring cyclophilin D activity by monitoring inhibition of the permeability transition (PT) pore by the decrease in absorbance at 540 nm of the suspension obtained in (3);
(5) preparation of a second suspension of isolated mitochondria and recombinant cyclophilin A in buffer solution;
(6) addition of the conjugate to the suspension obtained in (5);
(7) addition of Ca²⁺ to the suspension obtained in (6) to a concentration of 50 μM followed by immediate sedimentation of the mitochondria to provide a supernatant;
(8) monitoring cyclophilin A activity in the supernatant obtained in (7) by a standard spectrophotometric assay;
(9) separately determining dissociation constants (Kᵢ) for the conjugates of the invention with recombinant cyclophilin D and recombination cyclophilin A; and
(10) calculating the mitochondrial matrix/extramitochondrial accumulation ratio using the following equation:

$$\frac{50}{\begin{matrix}cyclophilin A inhibition ( \% ) at the concentration \\ of conjugate yielding 50 \% inhibition of PT pore\end{matrix}} \cdot \frac{{K}_{i} for cyclophilin D}{{K}_{i} for cyclophilin A}$$

Preferably in steps (1) and (5) mitochondria are isolated from rat liver by conventional procedures, such as that in Andreeva & Crompton (1994) Eur J Biochem 221, 261-268).

Preferably steps (1) to (10) are carried out at 25° C.

Preferably the Ca²⁺ in steps (3) and (7) is added as CaCl₂ and is added at a rate of 10 μM/min.

Preferably the mitochondria in step (7) are sedimented by centrifugation, for example in an Eppendorf bench centrifuge for one minute.

Preferably in step (8) the standard photometric analysis is that described by Kofron et al (1991) Biochemistry 30, 6127-6134.

Preferably, the suspensions obtained in steps (1) and (5) are identical.

Preferably said mitochondrial targeting group is a lipophilic cation or a mitochondrial targeting peptide.

Typically, the lipophilic cation is a phosphonium cation, an arsonium cation, an ammonium cation, flupritine, MKT-077, a pyridinium ceramide, a quinolium, a liposomal cation, a sorbitol guanidine, a cyclic guanidine, a rhodamine or a pyridine derivative.

Preferably, the lipophilic cation is a phosphonium cation, an arsonium cation, an ammonium cation, flupritine, MKT-077, a pyridinium ceramide, a quinolium, a liposomal cation, a sorbitol guanidine, a cyclic guanidine or a rhodamine.

Phosphonium cations and rhodamines are particularly preferred lipophilic cations.

Phosphonium, arsonium and ammonium cations are reviewed in Murphy et al (2007), Ann Rev. Pharm Toxicol. 47, 629-656. Typically a phosphonium, arsonium or ammonium cation is a cation of formula (II):

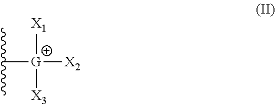

wherein G represents nitrogen, phosphorus or arsenic, and X₁, X₂ and X₃ independently represent alkyl, aryl, -alkylene-aryl or heteroaryl, wherein the alkyl and alkylene groups and moieties are unsubstituted or substituted by one or more, for example 1, 2 or 3, halogen atoms, hydroxyl, alkoxy or haloalkoxy groups, and the aryl and heteroaryl groups and moieties are unsubstituted or substituted by one, two or three halogen atoms, hydroxyl, alkoxy or haloalkoxy groups.

Preferably, said alkyl and alkylene groups and moieties are unsubstituted or substituted by one or more, preferably 1 or 2, halogen atoms. More preferably, said alkyl and alkylene groups and moieties are unsubstituted.

Preferably said aryl and heteroaryl groups and moieties are unsubstituted.

Preferably G represents a phosphorous or nitrogen atom, more preferably a phosphorous atom.

Preferably at least one of X₁, X₂ and X₃ represents phenyl or benzyl. More preferably all of X₁, X₂ and X₃ represent either phenyl or benzyl. Most preferably all of X₁, X₂ and X₃ represent phenyl or all of X₁, X₂ and X₃ represent benzyl.

Preferred cations of formula (II) are triphenylphosphonium (IIa) and tribenzylammonium (IIb):

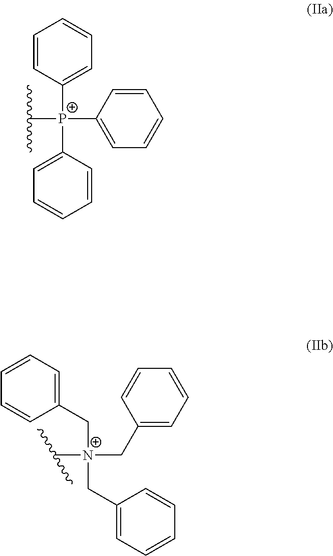

Flupritine and MKT-077 are described in Zimmer G, et al. Br J. Pharmacol. 1998, 123(6), 1154-8 and Modica-Napolitano et al, Cancer Res. 1996, 56, 544-550. Flupritine and MKT-077 have the following structures. They can be attached to the conjugate of the invention at any convenient position.

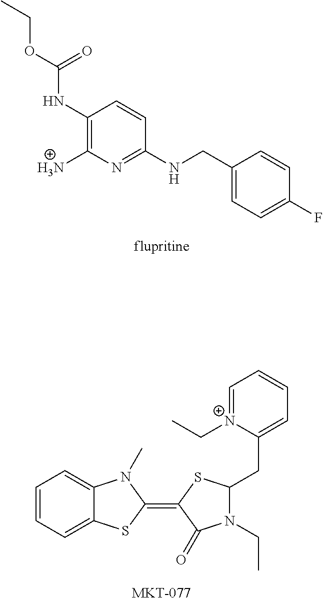

Pyridinium ceramides are described in Senkal et al, J Pharmacol Exp Ther. 317(3), 1188-1199. Typically, a pyridinium ceramide is compound of formula (IIIa) or (IIIb):

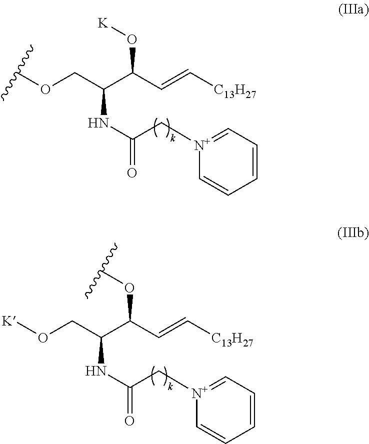

wherein K and K′ represent hydrogen or a protecting group, and k and k′ represent integers of 2 to 10.

Said protecting group may be any hydroxyl protecting group.

Preferably K and K′ represent hydrogen. Preferably k and k′ represent integers of 3 to 6, for example 4 or 5. More preferably K and K′ represent hydrogen and k and k′ represent 5.

Quinoliums are described in Weiss et al, Proc Natl Acad Sci USA, 84, 5444-5488. Typically, a quinolinium is di-cation of formula (IV):

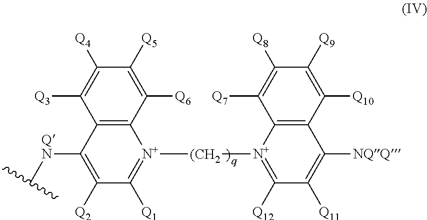

wherein Q₁ to Q₁₂ independently represent alkyl or hydrogen, Q′, Q″ and Q′″ independently represent alkyl or hydrogen and q represents an integer of 6 to 20, wherein said alkyl groups are unsubstituted or substituted by one or more halogen atoms, hydroxy, alkoxy or haloalkoxy groups.

Preferably, said alkyl groups are unsubstituted or substituted by one or more, preferably 1 or 2, halogen atoms, hydroxy or methoxy groups. More preferably, said alkyl groups are unsubstituted.

Preferably Q₁ to Q₁₂ independently represent methyl or hydrogen. Preferably Q′, Q″ and Q′″ represent hydrogen. Preferably q represents an integer of 8 to 14.

More preferably Q₁ and Q₁₂ represent methyl and Q₂ to Q₁₁ represent hydrogen. More preferably q represents 10. A dequalinium radical is preferred:

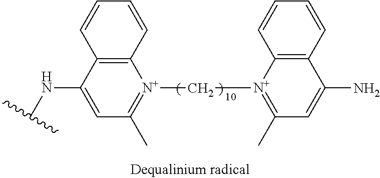

Liposomal cations are described in Souza et al, Mitochondrion 5 (2005) 352-358. A liposomal cation is a liposome-like cationic vesicle. Typically, a liposomal cation comprises a plurality of dequalinium molecules:

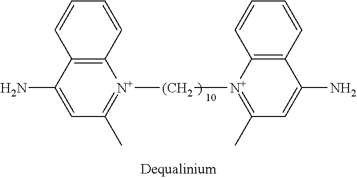

In this embodiment, the liposomal cation is typically linked non-covalently to the cyclosporin moiety.

Sorbitol guanidines are described in Maiti et al, Angew. Chem. Int. Ed. 2007, 46, 5880-5884. Typically, a sorbitol guanidine is a compound of formula (Va) to (Vf):

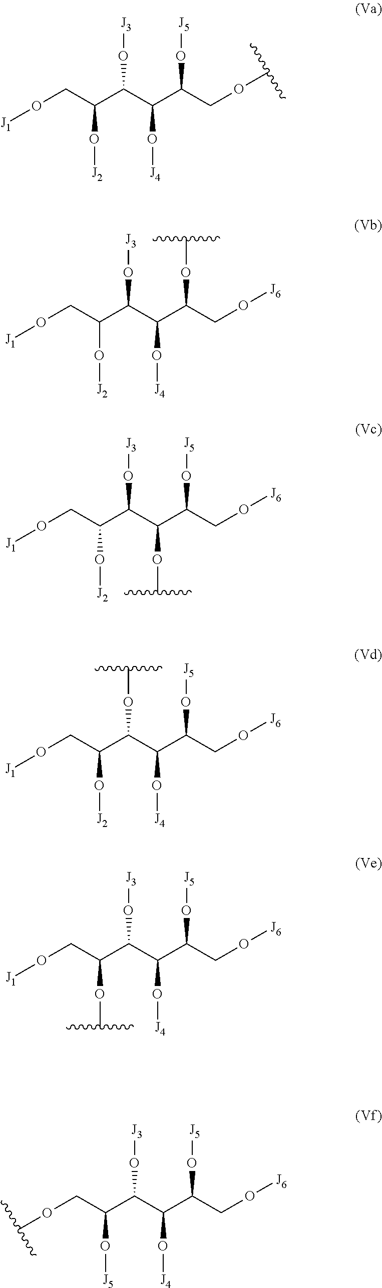

wherein J₁ to J₆ independently represent hydrogen, a protecting group, or a group of formula (Vg) or (Vh):

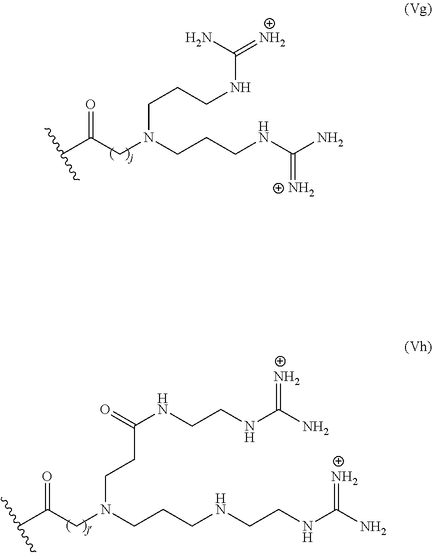

wherein j and j′ represent integers of 2 to 10, provided that at least one and preferably not more than four of J₁ to J₆ represent a group of formula (Vg) or (Vh).

Said protecting group may be any hydroxyl protecting group.

Preferably j and j′ represent integers of 4 to 8, for example 5 or 7.

In a preferred embodiment, said sorbitol guanidine is a compound of formula (Va), J₁ represents hydrogen or a protecting group, J₂ to J₅ represent groups of formula (Vg) and j represents 5 or 7.

In an alternative preferred embodiment, said sorbitol guanidine a compound is of formula (Va), J₁ represents hydrogen or a protecting group, J₂ to J₅ represent groups of formula (Vh) and j represents 5.

Cyclic guanidines are described in Kang et al, The Journal of Clinical Investigation, 119, 3, 454-464. Typically a cyclic guanidine is a compound of formula (VI):

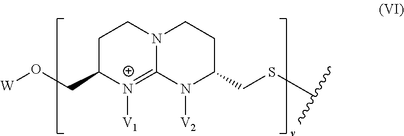

wherein W represents hydrogen or a protecting group, V₁ and V₂ independently represent hydrogen or alkyl and v is an integer of 1 to 6, wherein said alkyl groups are unsubstituted or substituted by one or more halogen atoms, hydroxy, alkoxy or haloalkoxy groups.

Preferably, said alkyl groups are unsubstituted or substituted by one or more, preferably 1 or 2, halogen atoms or hydroxy groups. More preferably, said alkyl groups are unsubstituted.

Said protecting group may be any hydroxyl protecting group.

Preferably W represents hydrogen or t-butyl-dimethyl-silyl (TBDMS). Preferably V₁ and V₂ represent hydrogen. Preferably v is an integer of 1 to 4, for example 1 or 2. More preferably W represents TBDMS, V₁ and V₂ represent hydrogen and v is 1.

Rhodamines are described in Hoye et al, Accounts of Chemical Research, 41, 1, 87-97. A rhodamine is typically a compound of formula (VII):

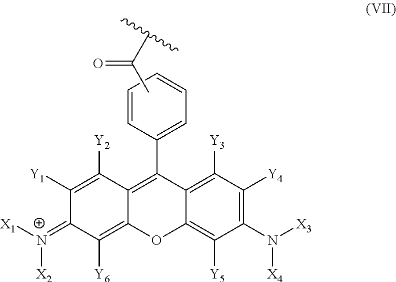

wherein X₁, X₂, X₃ and X₄ independently represent hydrogen or alkyl, and Y₁, Y₂, Y₃ and Y₄ independently represent hydrogen or alkyl, wherein said alkyl groups are unsubstituted or substituted by one or more halogen atoms, hydroxy, alkoxy or haloalkoxy groups.

Preferably, said alkyl groups are unsubstituted or substituted by one or more, preferably 1 or 2, halogen atoms or hydroxyl groups. More preferably, said alkyl groups are unsubstituted.

Preferably X₁, X₂, X₃ and X₄ independently represent hydrogen, methyl or ethyl.

Preferably Y₁, Y₂, Y₃ and Y₄ independently represent hydrogen or methyl.

Preferably the phenyl ring is substituted in the 2 or 4 position with the carbonyl moiety.

Preferred rhodamines include the following:

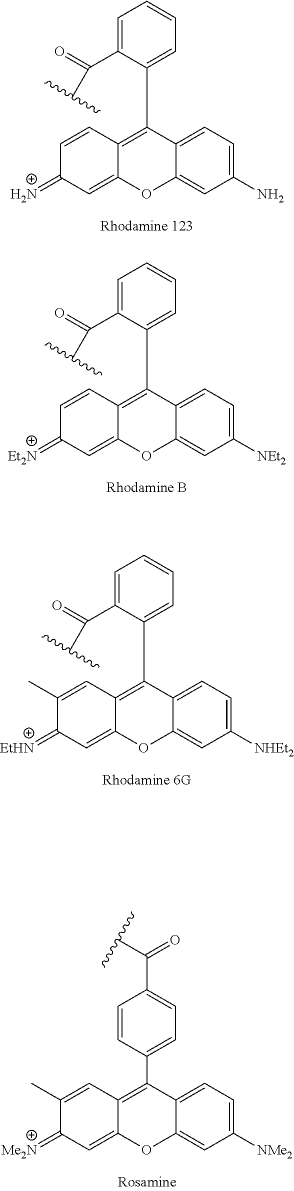

Rosamine is particularly preferred.

A pyridine derivative is typically a compound of formula (X):

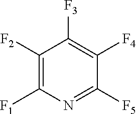

wherein F₁ to F₅ independently represent hydrogen, a halogen atom, —NO₂ or —NH₂. The pyridine derivative is typically attached to the cyclosporin moiety at any convenient position. For example, the pyridine derivative is preferably attached to the cyclosporin moiety via the nitrogen atom of the pyridine ring. Alternatively, when one of F₁ to F₅ represents —NH₂, the pyridine derivative is preferably attached to the cyclosporin moiety via the nitrogen atom amine moiety.

Preferably at least two of F₁ to F₅ represents hydrogen. Said NH₂ moiety may optionally be in the form of a tertiary ammonium cation associated with a pharmaceutically acceptable anion, for example a halide anion such as a chloride anion. Examples of pyridine derivatives include compounds of formula (Xa) and (Xb):

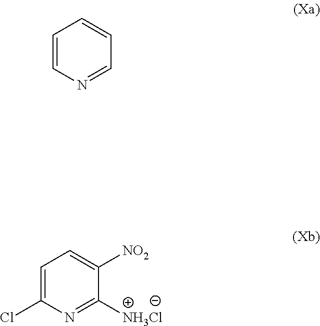

Mitochondrial targeting peptides are described in Horton et al, Chemistry and Biology 15, 375-382 and Hoye et al, Accounts of Chemical Research, 41, 1, 87-97. Typically a mitochondrial targeting peptide contains 4 to 16 amino acids. The amino acids are natural or unnatural amino acids. Typically, amino acids are selected from natural amino acids and diphenylalanine, cyclohexylalanine, hexylalanine, methylated tyrosine, dimethyltyrosine and napthylalanine. Said amino acids may be either the D- or L-enantiomers.

Preferred amino acids are basic amino acids and aromatic amino acids. Typical basic amino acids are lysine, arginine and glutamine, preferably lysine and arginine. Typical aromatic amino acids are phenylalanine, diphenyl alanine, cyclohexylalanine, hexylalanine, tyrosine, methylated tyrosine, dimethyltyrosine and napthylalanine.

A preferred class of mitochondrial targeting peptides are the SS tetrapeptides, which contain the structural motif of alternating aromatic and basic amino acids. Preferred aromatic residues in SS tetrapeptides are dimethyl tyrosine and phenylalanine. Preferred basic residues in SS tetrapeptides are arginine and lysine. Thus, an SS tetrapeptide is preferably a tetrapeptide containing alternating residues of (a) dimethyl tyrosine or phenylalanine, and (b) arginine or lysine.

Further preferred specific mitochondrial targeting peptides are those disclosed in Horton et al, Chemistry and Biology 15, 375-382 and Hoye et al, Accounts of Chemical Research, 41, 1, 87-97:
1. Fₓ-r-Fₓ-K-Fₓ-r-Fₓ-K
2. F-r-F-K-F-r-F-K
3. F-r-F_{X}-K-F-r-Fx-K
4. F-r-Y-K-F-r-Y-K
5. F_{X}-r-F_{X}-K
6. F-r-F-K
7. F-r-F_{X}K
8. F-r-F₂-K
9. F-r-Nap-K
10. F-r-Hex-K
11. F-r-Y_{Me}-K
12. F-r-F_{F}-K
13. F-r-Y-K
14. Y-r-Y-K
15. Y_{DM}-R-F-K
16. R-Y_{DM}-K-F
17. F-R-F-K

The following abbreviation are used above: F is phenylalanine, F₂ is diphenylalanine, F_{X} is cyclohexylalanine, Hex is hexylalanine, K is L-lysine, Nap is napthylalanine, R is L-arginine, r is D-arginine, Y is tyrosine, Y_{DM} is dimethyl tyrosine, Y_{Me} is methylated tyrosine and Q is glutamine.

Mitochondrial targeting peptides are typically attached to the cyclosporin moiety via either the C-terminus or the N-terminus of the peptide. The other end of the peptide is typically unprotected or protected with a suitable protecting group. Suitable protecting groups are well known to those skilled in the art.

Typically, the conjugate of the invention has the formula (I′):

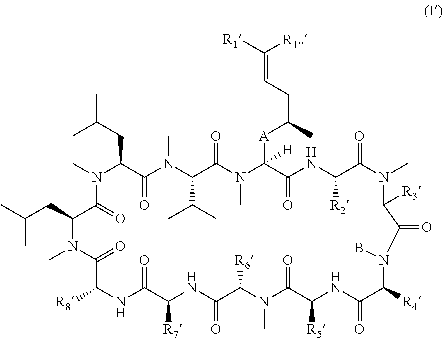

wherein:
one of R₁′ and R₁*′ represents methyl or -L₁-MTG₁ and the other represents hydrogen,
R₂′ represents R₂ as defined above or -L₂-MTG₂,
R₃′ represents R₃ as defined above or -L₃-MTG₃,
R₄′ represents R₄ as defined above or -L₄-MTG₄,
R₅′ represents isopropyl or -L₅-MTG₅,
R₆′ represents —CH₂CH(CH₃)CH₃ or -L₆-MTG₆,
R₇′ represents methyl or -L₇-MTG₇,
R₈′ represents methyl or -L₈-MTG₈, and
A and B are as defined above,
   wherein each of L₁ to L₈ independently represents a direct bond or a linker (L) as defined above, and each of MTG₁ to MTG₈ independently represents a mitochondrial targeting group as defined above, provided that at least one and not more than three of R₁′ or R₁*′ and R₂′ to R₈′ represent -L-MTG.

Preferably R₁′ represents methyl or -L₁-MTG₁ and R₁*′ represents hydrogen.

Preferably R₁′ represents methyl or -L₁-MTG₁, R₁*′ represents hydrogen, R₂′ represents R₂ as defined above, R₃′ represents R₃ as defined above or -L₃-MTG₃, R₄′ represents R₄ as defined above, R₅′ represents isopropyl, R₆′ represents —CH₂CH(CH₃)CH₃, R₇′ represents methyl, and R₈′ represents methyl.

In a preferred embodiment of the invention, R₁′ represents -L₁-MTG₁, R₁*′ represents hydrogen, R₂′ represents R₂ as defined above, R₃′ represents R₃ as defined above, R₄′ represents R₄ as defined above, R₅′ represents isopropyl, R₆′ represents —CH₂CH(CH₃)CH₃, R₇′ represents methyl, and R₈′ represents methyl.

In a further preferred embodiment of the invention, R₁′ represents methyl, R₁*′ represents hydrogen, R₂′ represents R₂ as defined above, R₃′ represents -L₃-MTG₃, R₄′ represents R₄ as defined above, R₅′ represents isopropyl, R₆′ represents —CH₂CH(CH₃)CH₃, R₇′ represents methyl, and R₈′ represents methyl.

Typically L₁ to L₈ independently represent a linker (L) as defined above.

Typically, L₁-MTG₁ is a compound of formula (VIII*):

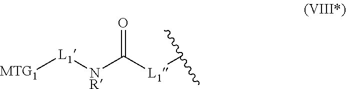

wherein L₁″ represents a direct bond or a phenylene moiety, L₁′ represents a straight chain C₁ to C₁₉ alkylene which is unsubstituted or substituted by one or more substituents selected from halogen atoms, hydroxy, alkoxy, alkyl, hydroxyalkyl, haloalkyl and haloalkoxy substituents, wherein 1 to 9 carbon atoms, preferably 1 to 4 carbon atoms, in said alkylene chain are replaced by spacer moieties selected from arylene, —O—, —NR′- and —C(O)NR′— moieties, wherein R′ is hydrogen or C₁ to C₆ alkyl, preferably hydrogen, and the arylene moiety is unsubstituted or substituted by one, two or three substituents selected from halogen atoms, hydroxy, alkyl or alkoxy groups.

Preferably, L₁-MTG₁ is a compound of formula (VIII):

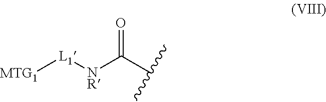

wherein L₁′ represents a straight chain C₁ to C₁₉ alkylene which is unsubstituted or substituted by one or more substituents selected from halogen atoms, hydroxy, alkoxy, alkyl, hydroxyalkyl, haloalkyl and haloalkoxy substituents, wherein 1 to 9 carbon atoms, preferably 1 to 4 carbon atoms, in said alkylene chain are replaced by spacer moieties selected from arylene, —O—, —NR′- and —C(O)NR′— moieties, wherein R′ is hydrogen or C₁ to C₆ alkyl, preferably hydrogen, and the arylene moiety is unsubstituted or substituted by one, two or three substituents selected from halogen atoms, hydroxy, alkyl or alkoxy groups.

Preferably, said straight chain C₁ to C₁₉ alkylene is unsubstituted or substituted by one or more, preferably 1 or 2, halogen atoms. Most preferably, said straight chain C₁ to C₁₉ alkylene is unsubstituted.

Preferably, the arylene spacer moiety is unsubstituted or substituted with one, two or three halogen atoms or hydroxy groups. When the arylene spacer moiety carries 2 or more substituents, the substituents may be the same or different. Most preferably the arylene spacer moiety is unsubstituted.

Preferably said spacer moieties comprise 0 to 1 arylene, 0 to 1 —O—, 0 to 1 —NH—, and 1 to 2 —C(O)NH— moieties.

More preferably L₁-MTG₁ is a compound of formula (VIIIa) or (VIIIb):

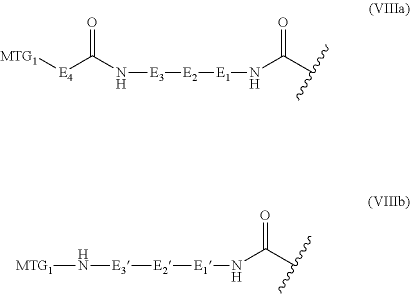

wherein E₁ and E₁′ represents unsubstituted straight chain C₁ to C₅ alkylene, E₂ and E₂′ represent a direct bond or —O—, E₃ and E₃′ represent unsubstituted straight chain C₁ to C₅ alkylene, and E₄ represents unsubstituted straight chain C₁ to C₆ alkylene.

E₁ and E₁′ preferably represent unsubstituted C₂ to C₄ alkylene. E₃ and E₃′ preferably represent unsubstituted C₂ to C₄ alkylene. E₄ preferably represents unsubstituted C₂ to C₆ alkylene.

Preferably L₁-MTG₁ is a compound of formula (VIIIa) when MTG₁ is a phosphonium cation, for example triphenylphosphonium, or L₁-MTG₁ is a compound of formula (VIIIb) when MTG₁ is a rhodamine, for example rosamine.

Alternatively, L₁-MTG₁ is preferably a compound of formula (VIII*a):

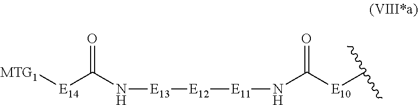

wherein E₁₀ represents a phenylene moiety, E₁₁ represents unsubstituted C₁ to C₄ alkylene, E₁₂ represent a direct bond or —O—, E₁₃ represents unsubstituted C₁ to C₄ alkylene and E₁₄ represents unsubstituted C₁ to C₆ alkylene.

Preferably E₁₁ represents unsubstituted C₂ to C₄ alkylene. Preferably E₁₃ represents unsubstituted C₂ to C₄ alkylene. E₁₄ preferably represents unsubstituted C₂ to C₅ alkylene.

Typical examples of an L₁-MTG₁ of formula (VIIIa) are the structures of formula (VIIIc) and (VIIId):

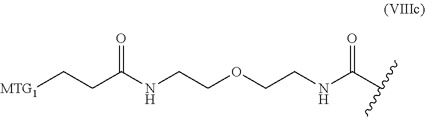

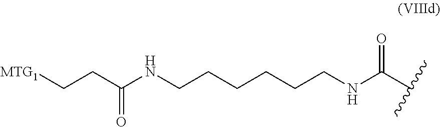

A typical example of an L₁-MTG₁ of formula (VIII*a) is the structure of formula (VIIIe):

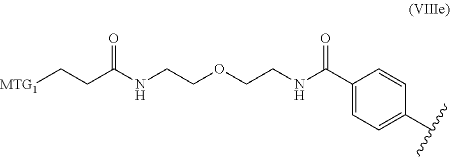

Preferably, L₃-MTG₃ is a compound of formula (IX):

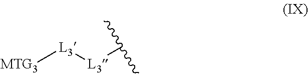

wherein L₃″ represents unsubstituted straight chain C₁ to C₂ alkylene and L₃′ represents C₁ to C₁₈ alkylene which is unsubstituted or substituted by one or more substituents selected from halogen atoms, hydroxy, alkoxy, alkyl, hydroxyalkyl, haloalkyl and haloalkoxy substituents, wherein 1 to 10 carbon atoms, preferably 1 to 4 carbon atoms, in said C₁ to C₁₈ alkylene chain are replaced by spacer moieties selected from arylene, —O—, —NR′— and —C(O)NR′— moieties, wherein R′ is hydrogen or C₁ to C₆ alkyl, preferably hydrogen, and the arylene moiety is unsubstituted or substituted by one, two or three substituents selected from halogen atoms, hydroxy, alkyl or alkoxy groups.

Preferably, said straight chain C₁ to C₁₈ alkylene is unsubstituted or substituted by one or more, preferably 1 or 2, halogen atoms. Most preferably, said straight chain C₁ to C₁₈ alkylene is unsubstituted.

Preferably, the arylene spacer moiety is unsubstituted or substituted with one, two or three halogen atoms or hydroxy groups. When the arylene spacer moiety carries 2 or more substituents, the substituents may be the same or different. Most preferably the arylene spacer moiety is unsubstituted.

Preferably said spacer moieties comprise 0 to 1 arylene, 0 to 1 —O—, 0 to 1 —NH— and 1 to 2—C(O)NH— moieties.

Preferably L₃-MTG₃ is a compound of formula (IXa) or (IXb):

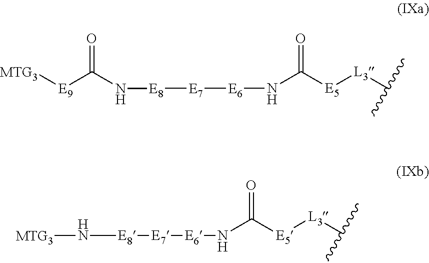

wherein E₅ and E₅′ represent a direct bond or unsubstituted arylene, E₆ and E₆′ represent unsubstituted C₁ to C₄ alkylene, E₇ and E₇′ represent a direct bond or —O—, E₈ and E₈′ represent unsubstituted C₁ to C₄ alkylene and E₉ represents unsubstituted C₁ to C₆ alkylene.

Preferably E₅ and E₅′ represent unsubstituted arylene, more preferably unsubstituted phenylene. Preferably E₇ and E₇′ represent unsubstituted C₂ to C₄ alkylene. Preferably E₈ and E₈′ represent unsubstituted C₂ to C₄ alkylene. E₉ preferably represents unsubstituted C₂ to C₅ alkylene.

Preferably L₃-MTG₃ is a compound of formula (IXa) when MTG₃ is a phosphonium cation, for example triphenylphosphonium, or L₃-MTG₃ is a compound of formula (IXb) when MTG₃ is a rhodamine, for example rosamine.

Typical examples of an L₃-MTG₃ of formula (IXa) are the structures of formula (IXc), (IXe) and (IXf). A typical example of an L₃-MTG₃ of formula (IXb) is the structure of formula (IXd).

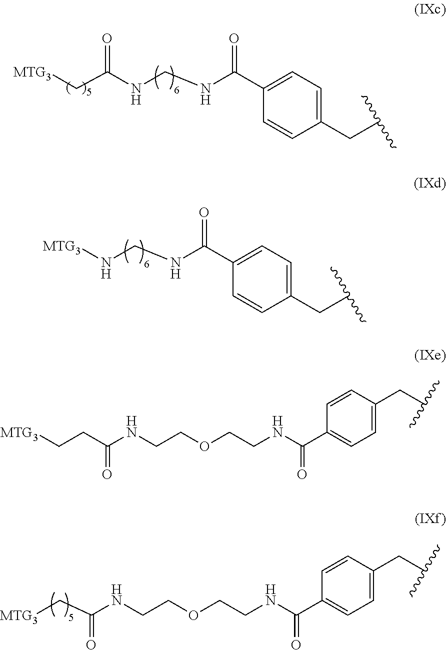

In a particularly preferred embodiment of the invention:
R₁′ represents -L₁-MTG₁, R₁*′ represents hydrogen, R₂′ represents R₂ as defined above, R₃′ represents R₃ as defined above, R₄′ represents R₄ as defined above, R₅′ represents isopropyl, R₆′ represents —CH₂CH(CH₃)CH₃, R₇′ represents methyl, and R₈′ represents methyl, and
L₁-MTG₁ is a compound of formula (VIIIa):

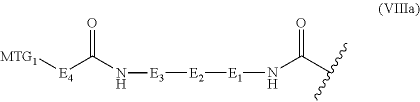

wherein E₁ to E₄ are as defined above and MTG₁ represents triphenylphosphonium, or
L₁-MTG₁ is a compound of formula (VIIIb):

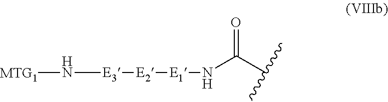

wherein E₁′ to E₃′ are as defined above and MTG₁ represents rosamine.

In another particularly preferred embodiment of the invention:
R₁′ represents -L₁-MTG₁, R₁*′ represents hydrogen, R₂′ represents R₂ as defined above, R₃′ represents R₃ as defined above, R₄′ represents R₄ as defined above, R₅′ represents isopropyl, R₆′ represents —CH₂CH(CH₃)CH₃, R₇′ represents methyl, and R₈′ represents methyl, and
L₁-MTG₁ is a compound of formula (VIII*a):

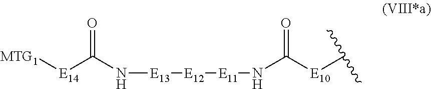

wherein E₁₀ to E₁₄ are as defined above and MTG₁ represents triphenylphosphonium

In yet another particularly preferred embodiment of the invention:
R₁′ represents methyl, R₁*′ represents hydrogen, R₂′ represents R₂ as defined above, R₃′ represents -L₃-MTG₃, R₄′ represents R₄ as defined above, R₅′ represents isopropyl, R₆′ represents —CH₂CH(CH₃)CH₃, R₇′ represents methyl, and R₈′ represents methyl, and
L₃-MTG₃ is a compound of formula (IXa):

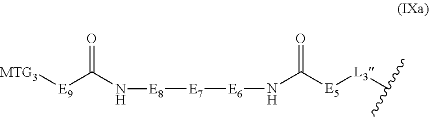

wherein L₃″ and E₅ to E₉ are as defined above and MTG₃ represents triphenylphosphonium or
L₃-MTG₃ is a compound of formula (IXb):

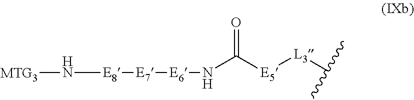

wherein L₃″ and E₅′ to E₈′ are as defined above and MTG₃ represents rosamine.

Particularly preferred conjugates of the invention are compounds of formula (I′a), (I′b), (I′c), (I′d), (I′e), (I′f) and (I′g) and pharmaceutically acceptable salts thereof:

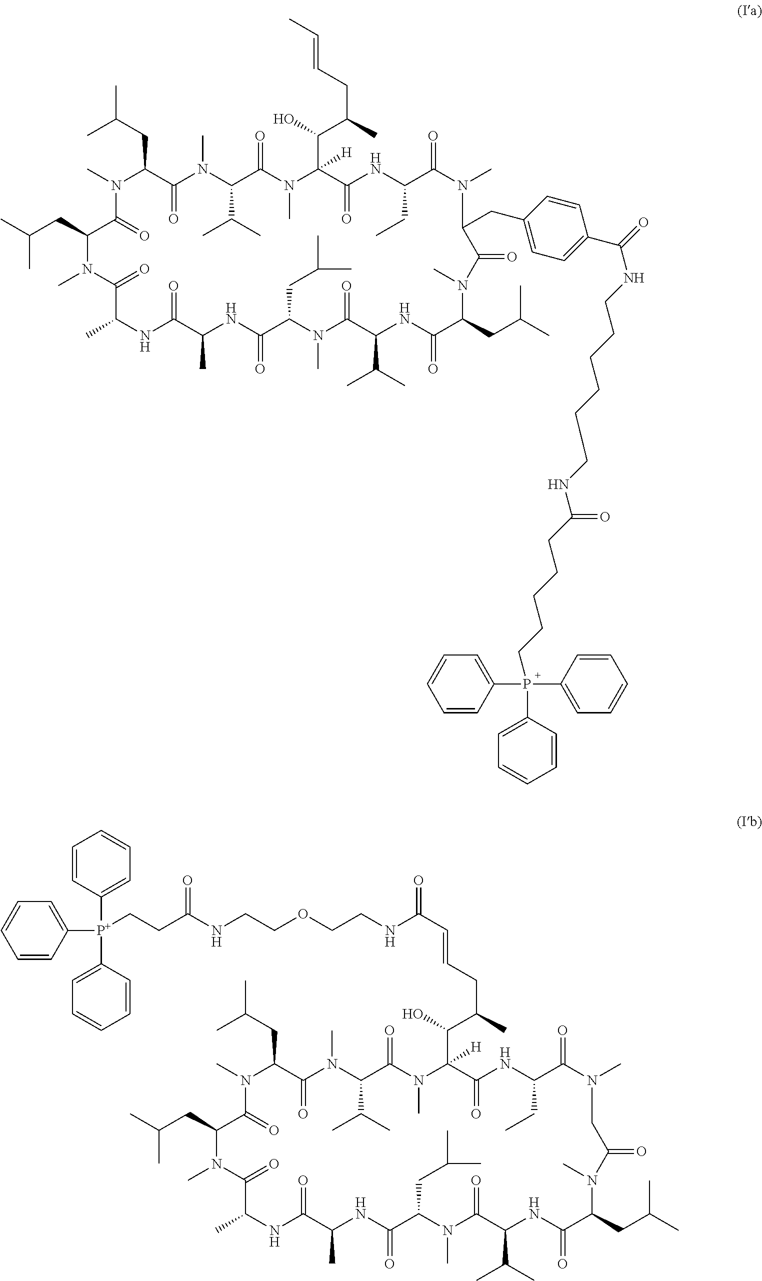

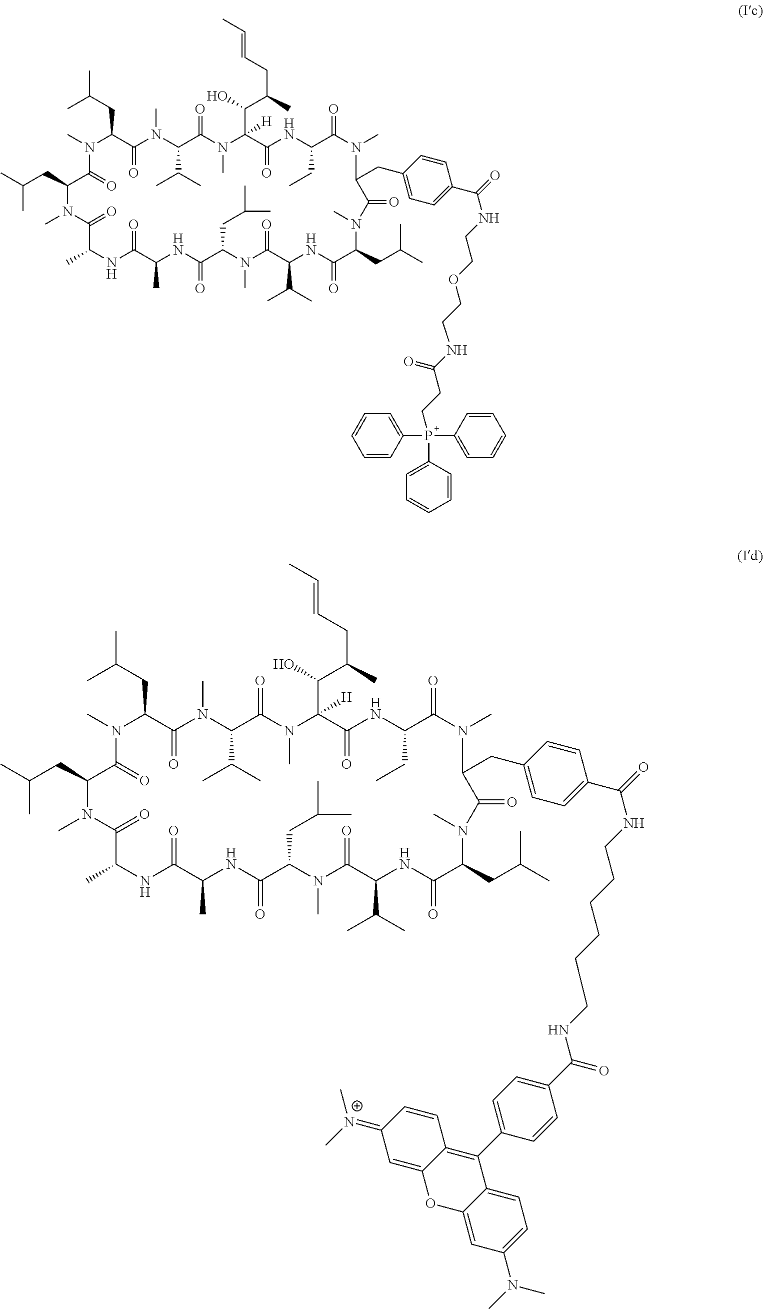

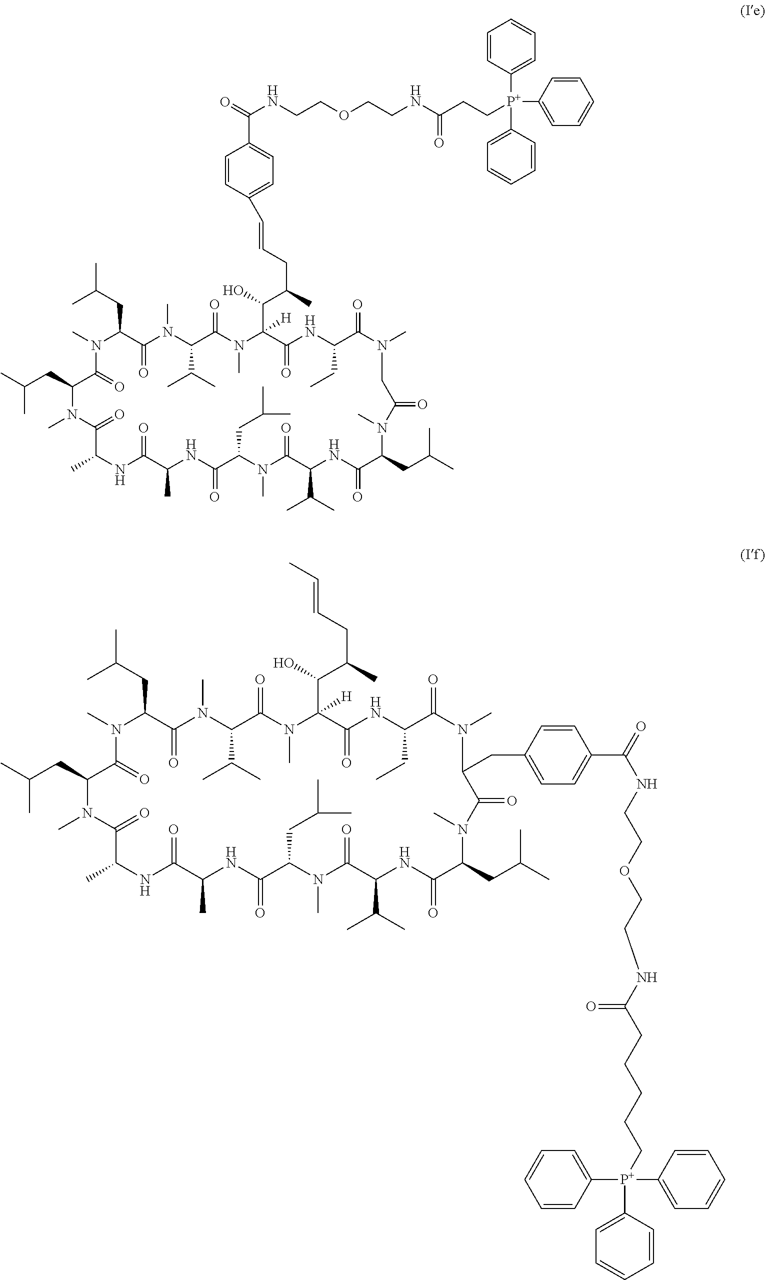

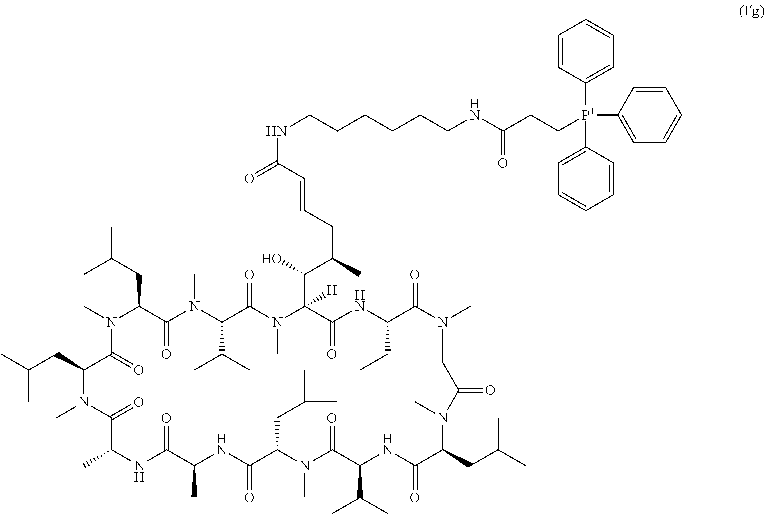

As used herein, an “alkyl” group or moiety is typically a C₁₋₂₀ alkyl, preferably a C₁₋₁₂ alkyl, more preferably a C₁₋₆ alkyl and most preferably a C₁₋₃ alkyl. Particularly preferred alkyl groups and moieties include, for example, methyl, ethyl, propyl, isopropyl, butyl, isobutyl, tert-butyl, pentyl and hexyl.

As used herein, an alkylene group is a said alkyl group which is divalent.

As used herein, an alkoxy group is a said alkyl group which is attached to an oxygen atom. The alkoxy group is typically a C₁₋₂₀ alkoxy group, preferably a C₁₋₁₂ alkoxy group, more preferably a C₁₋₆ alkoxy group and most preferably a C₁₋₃ alkoxy group. Particularly preferred alkoxy groups include, for example, methyoxy, ethyoxy, propoxy, isopropoxy, butoxy, isobutoxy, tert-butoxy, pentoxy and hexoxy.

As used herein, a halogen is typically chlorine, fluorine, bromine or iodine and is preferably chlorine, bromine or fluorine.

A haloalkyl or haloalkoxy group is typically a said alkyl or alkoxy group substituted by one or more said halogen atoms. Typically, it is substituted by 1, 2 or 3 said halogen atoms. Preferred haloalkyl and haloalkoxy groups include perhaloalkyl and perhaloalkoxy groups such as —CX₃ and —OCX₃ wherein X is a said halogen atom, for example chlorine and fluorine. Particularly preferred haloalkyl groups are —CF₃ and —CCl₃. Particularly preferred haloalkoxy groups are —OCF₃ and —OCCl₃.

A hydroxyalkyl group is typically a said alkyl group substituted by one or more hydroxy groups, preferably 1, 2 or 3 hydroxy groups, more preferably 1 hydroxy group.

As used herein, the term “aryl” is a C₆₋₁₀ monoaromatic or polyaromatic system, wherein said polyaromatic system may be fused or unfused. Examples of aryl groups are phenyl, and naphthyl. Phenyl is preferred.

As used herein, an arylene group is a said aryl group which is divalent. Phenylene is preferred. A said phenylene group may be divalent in the 1, 2 or 1, 3 or 1,4 positions. 1,4 phenylene is preferred.

As used herein, the term “heteroaryl” is a 5- to 6-membered ring system containing at least one heteroatom, preferably 1 or 2 heteroatoms, selected from O, S and N. Examples of heteroaryl groups are pyridyl, pyrazinyl, pyrimidinyl, pyridazinyl, furyl, oxadiazolyl, oxazolyl, imidazolyl, thiazolyl, thiadiazolyl, thienyl, pyrrolyl, pyridinyl, triazolyl, tetrazolyl, and pyrazolyl groups.

The term “-alkylene-aryl” refers to a said alkylene group attached to a said aryl group. A typical -alkylene-aryl group is benzyl.

As used herein, the term protecting group refers to any moiety that protects a functional group such as an alcohol, amine or carboxylic acid. An hydroxyl protecting group is preferably a trialkylsilyl, such as trimethyl-silyl (TMS) or t-butyl-dimethyl-silyl (TBDMS), tetrahydropyranyl (THP), benzyl (Bn), methyl (Me), acetyl (Ac) or benzoyl (Bz). An amine protecting group is preferably carbobenzyloxy (Cbz) or benzyl (Bn). A carboxylic acid is preferably protected as an ester, such as a methyl ester, benzyl ester, t-butyl ester or silyl ester.

As used herein, a pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases such as alkyl amines, aralkyl amines or heterocyclic amines.

The present invention also includes the use of solvate forms of the conjugates of the invention. The terms used in the claims encompass these forms.

The invention furthermore relates to the conjugates of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

The invention further includes the compounds of the present invention in prodrug form. Such prodrugs are generally compounds of the invention wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include esters, wherein the reversion may be carried out be an esterase etc. Other such systems will be well known to those skilled in the art.

The conjugates of the invention may be prepared by standard methods known in the art. Compounds of formula (I) are known compounds which are commercially available.

Compounds of formula (I) can then be linked to mitochondrial targeting groups using standard techniques known in the art.

For example, a specific conjugate of the invention (Compound 1) can be conveniently prepared as shown in Scheme 1. This pathway starts with commercially available cyclosporin A and proceeds via intermediates 1 and 2 over multiple steps. Suitable reagents for each step are: (i) lithium diisopropylamide, trimethylsilyl chloride, 4-bromomethylbenzoate, ii) LiOH, methanol, iii) Fmoc-diaminohexane, PyBOP, iv) piperidine, DMF, v) 5-(carboxypentyl)triphenylphosphonium bromide, PyBOP.

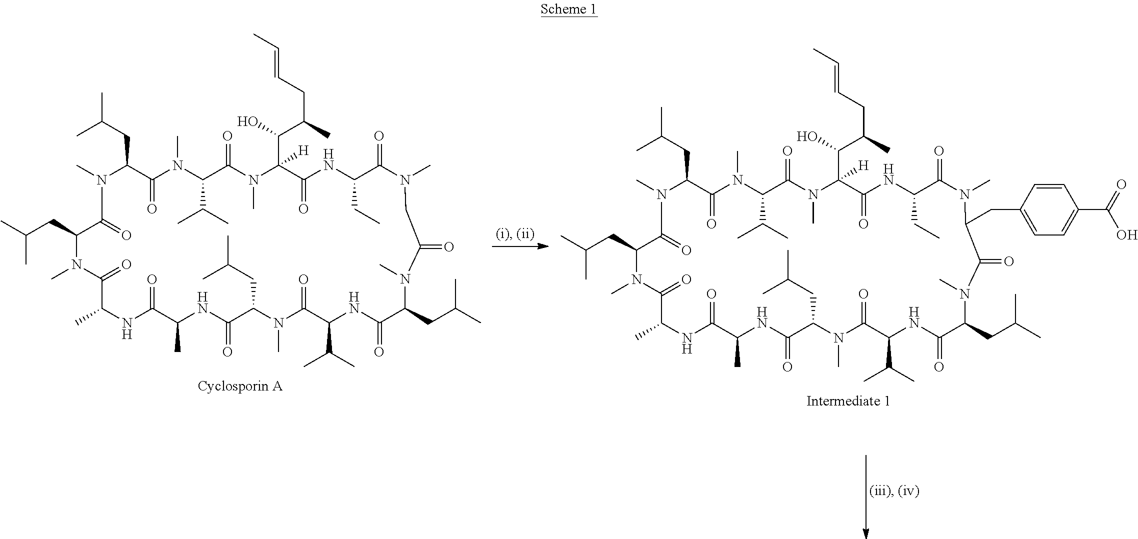

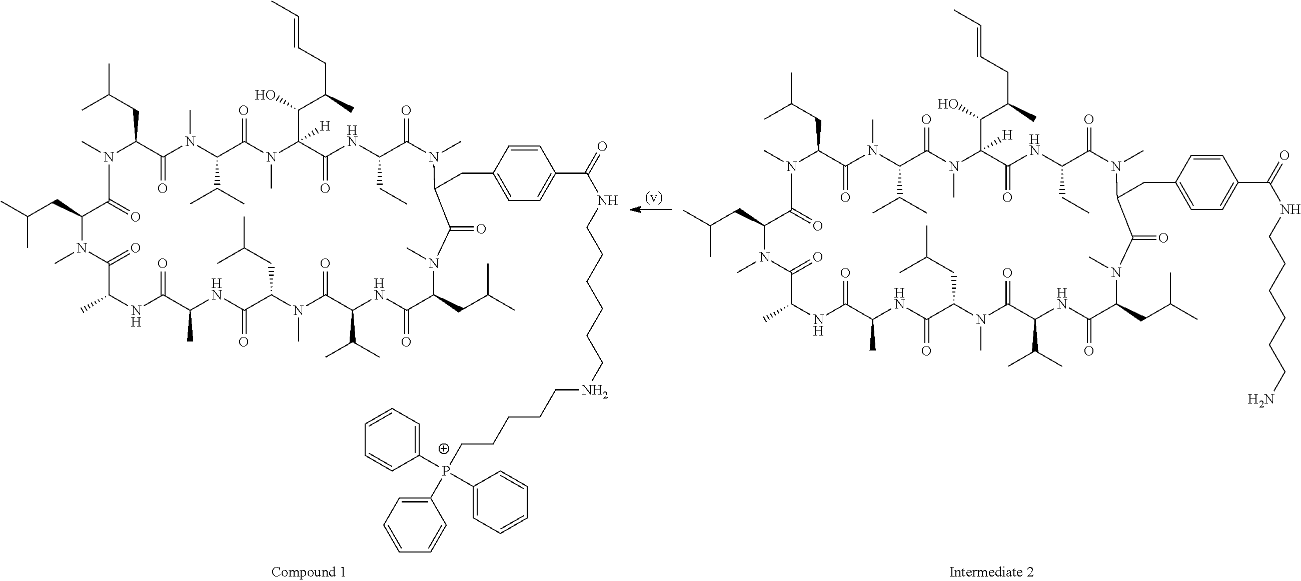

The conjugates of the invention are useful in the treatment or prevention of diseases or disorders susceptible to amelioration by inhibition of cyclophilin D, particularly in humans. Thus, the conjugates of the invention may preferably be used to improve the condition of a patient who has suffered from, is suffering from or is at risk of suffering from ischaemia/reperfusion injury. In particular, the compounds of the invention may be used in the treatment of cerebral or myocardial ischaemia/reperfusion injury. Neurodegenerative diseases, such as Alzheimer's disease and multiple sclerosis may also be treated by inhibition of cyclophilin D.

Thus, the present invention further provides a conjugate of the invention for use in the treatment of the human or animal body.

The present invention further provides a conjugate of the invention for use in the treatment or prevention of a disease or disorder susceptible to amelioration by inhibition of cyclophilin D.

The present invention further provides use of a conjugate of the invention in the manufacture of a medicament for use in the treatment of a disease or disorder susceptible to amelioration by inhibition of cyclophilin D.

The present invention further provides a method of treating a patient suffering from or susceptible to disease or disorder susceptible to amelioration by inhibition of cyclophilin D, which method comprises administering to said patient a conjugate of the invention.

Preferably said disease or disorder susceptible to amelioration by inhibition of cyclophilin D is ischaemia/reperfusion injury or a neurodegenerative disease. Examples of neurodegenerative diseases include Alzheimer's disease and multiple sclerosis. Most preferably however said disease or disorder susceptible to amelioration by inhibition of cyclophilin D is ischaemia/reperfusion injury.

The conjugates of the invention may be administered to humans in various manners such as oral, rectal, vaginal, parenteral, intramuscular, intraperitoneal, intraarterial, intrathecal, intrabronchial, subcutaneous, intradermal, intravenous, nasal, buccal or sublingual routes of administration. The particular mode of administration and dosage regimen will be selected by the attending physician, taking into account a number of factors including the age, weight and condition of the patient.

The pharmaceutical compositions that contain the conjugates of the invention as an active principal will normally be formulated with an appropriate pharmaceutically acceptable excipient, carrier or diluent depending upon the particular mode of administration being used. For instance, parenteral formulations are usually injectable fluids that use pharmaceutically and physiologically acceptable fluids such as physiological saline, balanced salt solutions, or the like as a vehicle. Oral formulations, on the other hand, may be solids, e.g. tablets or capsules, or liquid solutions or suspensions.

Thus, the present invention also provides a pharmaceutical composition comprising a conjugate of the invention and a pharmaceutically acceptable excipient, diluent or carrier.

Compositions may be formulated in unit dosage form, i.e., in the form of discrete portions containing a unit dose, or a multiple or sub-unit of a unit dose.

The amount of the conjugate of the invention that is given to a patient will depend upon on the activity of the particular conjugate in question. Further factors include the condition being treated, the nature of the patient under treatment and the severity of the condition under treatment. The timing of administration of the conjugate should be determined by medical personnel, depending on whether the use is prophylactic or to treat ischemia/reperfusion injury. As a skilled physician will appreciate, and as with any drug, the conjugate may be toxic at very high doses. For example, the agent may be administered at a dose of from 0.01 to 30 mg/kg body weight, such as from 0.1 to 10 mg/kg, more preferably from 0.1 to 5 mg/kg body weight.

The conjugates of the invention may be given alone or in combination with one or more additional active agents useful for treating a disease or disorder susceptible to amelioration by inhibition of cyclophilin D, such as ischaemia/reperfusion injury or a neurodegenerative disease. Two or more active agents are typically administered simultaneously, separately or sequentially. The active ingredients are typically administered as a combined preparation.

The conjugates of the invention can also be used as reagents. For example, they are useful in non-therapeutic experimental procedures in which selective inhibition of cyclophilin D is required. The conjugates of the invention are therefore useful as laboratory reagents for assessing the involvement of cyclophilin D in cellular processes, such as cell death. No such reagents are currently available. Typically, said non-therapeutic experimental procedure is an assay. Thus, the invention also provides a non-therapeutic use of a conjugate of the invention as a reagent for an experimental assay.

The following Examples illustrate the invention.

### EXAMPLES

### Materials and Methods

Preparation of Recombinant Cyclophilin D (cyP-D) and Cyclophilin A (CyP-A)

Recombinant rat CyP-D was prepared and purified as described previously in Li et al, Biochem. J. 383, 101-109. For CyP-A, the coding sequence in rat was PCR-amplified with the addition of BamH1 and EcoR1 restriction sites, and cloned between the same sites of pGEX-4T-1 in E coli DH5α cells. Transformed cells were grown for 5 hours at 21° C. The GST/CyP-A fusion protein was extracted, purified on GSH sepharose, and then cleaved with thrombin to release CyP-A. The CyP-A was purified on cation exchange (Mono-S) and gel filtration (Superdex-75) columns to give a single band on SDS-PAGE.

Interactions of Cyclosporin and Cyclosporin Conjugates with Cyclophilins and Calcineurin

Dissociation constants for cyclophilin/cyclosporin and cyclophilin/cyclosporin conjugate interactions were measured as inhibitor constants, Kᵢ. PPIase assays were conducted at 15° C. in 100 mM NaCl/20 mM Hepes (pH 7.5) using N-succinyl-alanyl-alanyl-prolyl-4-nitroanilide as test peptide as described in McGuinness et. al. (1990) Eur. J. Biochem. 194, 671-679. The peptide contains a mixture of cis and trans Ala-Pro isomers, of which only the trans conformer is hydrolysed by chymotrypsin at the C-terminal amide bond to release chromophore. Existing trans isomer is cleaved within the mixing time; further cleavage requires cis-trans isomerisation, which is measured. Cyclophilins were preincubated with cyclosporins for 5 min before addition of chymotrypsin and 60 μM peptide (containing about 35 μM cis peptide) to start the reaction.

Cyclosporins inhibit by competing at the active site with substrate. Accordingly, kinetic data were analysed by the Henderson equation for a tight binding, competitive inhibitor, which can be written:

$$\frac{{I}_{o}}{P} = \frac{1}{( 1 - P )} \cdot {K}_{i} \cdot ( 1 + \frac{S}{{K}_{M}} ) + {E}_{o}$$

Where Eₒ and Iₒ are the total concentrations of enzyme and inhibitor (cyclosporin) respectively, Kᵢ is the enzyme/inhibitor dissociation constant, K_{M} is the Michaelis constant, and S is the substrate concentration. P is the fractional inhibition, equal to {1−(vᵢ/vₒ)}, where vᵢ and vₒ are the reaction velocities in the presence and absence of inhibitor, respectively.

The K_{M} value for the cis peptide used is much higher than its concentration in the assay (<35 μM). Since K_{M}>>S the equation may be simplified:

$$\frac{{I}_{o}}{P} = \frac{1}{( 1 - P )} \cdot {K}_{i} + {E}_{o} \qquad Equation 1$$

And plots of Iₒ/P against 1/(1−P) are linear with slope=Kᵢ

Interaction of cyclophilin/cyclosporin and cyclophilin/cyclosporin conjugate complexes with calcineurin was evaluated from inhibition of the phosphatase activity of calcineurin as measured by the release of inorganic phosphate from the RII phosphopeptide (Biomol International UK, Exeter, UK).

Experiments with Isolated Mitochondria

Mitochondria were isolated from rat livers as described before (Crompton et al, Eur J. Biochem 178, 489-501). PT pore opening was monitored by the associated swelling of the mitochondria as measured by the decrease in absorbance at 540 nm. Mitochondria (2 mg of protein) were suspended in 3 ml of 120 mM KCl/2 mM KH₂PO₄/3 mM succinate/10 mM Hepes (pH 7.2)/1 μM rotenone/5 μM EGTA/recombinant CyP-A (1 μg) and test cyclosporins, and maintained under continuous stirring at 25° C. After 5 min, CaCL₂ was slowly infused (10 μM/min) to a final concentration of 50 μM. In a parallel incubation, mitochondria were sedimented immediately after Ca²⁺ addition and the CyP-A activity of the supernatant determined.

### Neuronal Cultures and Assays

B50 cells from a rat neuronal cell line and a clone stably overexpressing CyP-D were cultured on coverslips in DMEM (Dulbecco's minimal essential medium) containing 10% foetal calf serum. Uptake of the fluorescent tetramethylrhodamine ethyl ester (TMRE) was measured by incubating the cells at 25° C. in basic medium (140 mM NaCl/4 mM KCl/24 mM Hepes (pH 7.4)/1 mM MgSO₄/1 mM CaCl₂/1 mM KH₂PO₄/11 mM glucose) containing 50 nM TMRE.

Fluorescence images (530 nm />595 nm) were obtained with an Olympus IX-70 fluorescence microscope with X60 oil objective, Micromax 1401E CCD camera and Metamorph software (Universal imaging). For nitroprusside treatment, cells were incubated in basic medium containing 100 μM sodium nitroprusside for 40 min and then returned to DMEM medium. After 5 hr, cells were extracted and extracts assayed for caspase-3 activity using the fluorescent 7-amino-4-trifluoromethylcoumarin (AFC) derivative of the caspase-3/-7 selective substrate (Ac-DEVD-AFC) as described in Capano et al, Biochem J. (2002) 363, 29-36.

For antisense suppression of CyP-A, cells were incubated with 1 μM phosphorothioate ODN 5′-CATGGCTTCCACAATGCT for 48 hours as described in Capano et al, Biochem J. (2002) 363, 29-36.

Hippocampal neurons were prepared from 2-4 day old Sprague Dawley rats as mixed cultures with glial cells. Dissected hippocampi were incubated in Hanks balanced salt solution (HBSS) containing 0.1% w/v trypsin for 5 min at 37° C., followed by two washes in HBSS. Hippocampi were then dissociated in HBSS containing 1 mg/ml BSA, 5% foetal calf serum and 8 mM MgCl₂.

Dissociated cells were sedimented, suspended in Neurobasal A medium (NBA) supplemented with 0.5 mM glutamine, 2% B27 supplement (Gibco) and 5% foetal calf serum, seeded onto coverslips, and incubated under 95% air/5% CO₂ in the same medium plus antimitotics mix (5-fluor-2′-deoxyuridine, uridine, 1-beta-D-arabinofuranosylcytosine, 1 μM of each). Medium minus antimitotics was introduced after 3 days.

For oxygen and glucose deprivation (OGD), coverslips with hippocampal neurons were seated to form the base of a small, capped chamber mounted on the microscope stage. The chamber contained an inlet and outlet for continuous gassing, input and output tubes for changing the incubation medium, and a heating element to maintain the temperature at 36° C. Pseudo-ischaemic conditions were imposed by omitting glucose and displacing air with N₂ in the experimental chamber.

Cells were incubated under 95% N₂/5% CO₂ with (pregassed) 145 mM NaCl/26 mM NaHCO₃/5 mM KCl/1.8 mM CaCl₂/0.8 mM MgCl₂/4 μM ethidium homodimer/2 μM Hoechst 33342 and cyclosporins as indicated. After 30 min the gassing was switched to 95% air/5% CO₂ and the medium replaced with NBA medium containing 4 μM ethidium homodimer. Hippocampal neurons were identified under brightfield illumination and then correlated with their respective nuclei (just above the focal plane of glia nuclei) from Hoechst fluorescence.

Necrosis was quantified from nuclear staining by fluorescent ethidium homodimer, which is live-cell impermeant, but enters dead cells. For treatment with glutamate, cultures were incubated under 95% air/5% CO₂ in 150 mM NaCl/5 mM KCl/25 mM NaHCO₃/2.3 mM CaCl₂/6 mM Glucose/5 mM Hepes (Lockes medium) containing cyclosporins (as indicated). After 10 min, 1 mM glutamate was added. After a further period (as indicated), cells were returned to NBA medium containing Hoechst 33342 and ethidium homodimer, and necrosis was quantified 15 min later.

Statistical analyses were made using a one-way ANOVA test with a post-test of Dunnett.

### Heart Cell Culture and Assays

Ventricular cardiomyocytes were prepared from 14-day old Sprague-Dawley rats and seeded on to glass coverslips as described in Doyle et al, Biochem J. (1999) 341, 127-132. Cells were cultured under CO₂/air (1:19) at 37° C. in M199 medium (Sigma) containing 20 units/ml penicillin, 2 μg/ml vitamin B₁₂ and 10% (w/v) foetal calf serum.

Ischaemia/reperfusion was mimicked by transient oxygen and glucose deprivation followed by glucose-replete normoxia. For oxygen and glucose deprivation, coverslips with cardiomyocytes were incubated under O₂-free N₂ in 145 mM NaCl/4 mM KCl/24 mM Hepes (pH 7.4)/1.8 mM CaCl₂/1 mM MgCl₂/1 mM KH₂PO₄/4 μM ethidium homodimer/2 μM Hoechst 33342 and cyclosporins as indicated. After 4 hours, 10 mM glucose and 50 μM t-butylhydroperoxide were added and the cells reoxygenated by switching the gassing to air. Necrosis was determined from the staining of cell nuclei by ethidium homodimer. Results are given as means±SEM (n=4).

### Synthesis of Compound 1

Compound 1 was prepared according to Scheme 1 above, via Intermediates 2 and 3.

### Intermediate 1

4-(((5S,8S,11S,14S,17R,20S,23S,26S,29S,32S)-32-ethyl-29-((1R,2R,E)-1-hydroxy-2-methylhex-4-enyl)-5,11,20,23-tetraisobutyl-8,26-diisopropyl 1,4,10,14,17,19,22,25,28-nonamethyl-3,6,9,12,15,18,21,24,27,30,33-undecaoxo-1,4,7,10,13,16,19,22,25,28,31-undecaazacyclotritriacontan-2-yl)methyl)benzoic acid

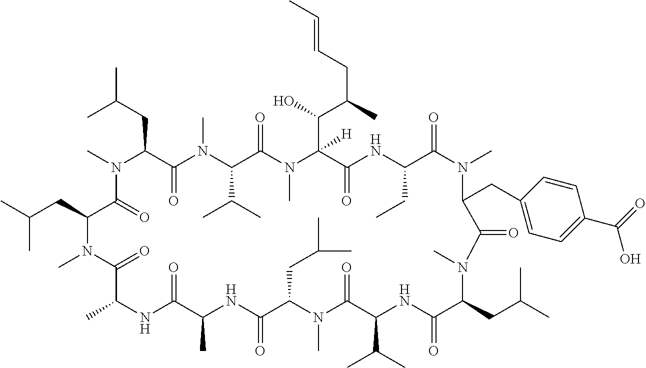

To a stirred solution of cyclosporine A (1.00 g, 0.83 mmol) in dry THF (25 ml) under nitrogen at 0° C. is added dropwise fresh LDA (4.6 mmol, 2.3 ml, 2M in THF). To the resultant deep brown suspension is added, dropwise, trimethylsilylchloride (0.83 mmol, 0.1 ml) to give a clear brown solution. The mixture is stirred at 0° C. for 10 minutes. Then a further LDA (7.1 mmol, 3.5 ml, 2M in THF) was added dropwise and the reaction stirred for 30 minutes at 0° C.

A solution of 4-Bromomethylbenzoate (1.3 g, 5.8 mmol) in dry THF (10 ml) was added dropwise to give a pale yellow solution which is stirred for a further 1 hour. The reaction is quenched with saturated aqueous ammonium chloride (10 ml) followed by 2M hydrochloric acid and then diluted with CH₂Cl₂ (20 ml). The separated aqueous layer is extracted with CH₂Cl₂ (2×20 ml). The combined organic phases are washed with 2M HCl(aq) (2×20 ml), saturated NH₄Cl(aq) (2×20 ml) and brine (2×20 ml) and then dried (MgSO₄(s)).

The volatiles were removed al vacuo to leave a dark brown oil residue. Purification by flash chromatography eluting with 6% MeOH in CH₂Cl₂ gave a yellow solid residue (0.930 g) as a mixture of the unreacted cyclosporine and the alkylated ester product: methyl 4-(((5S,8S,11S,14S,17R,20S,23S,26S,29S,32S)-32-ethyl-29-((1R,2R,E)-1-hydroxy-2-methylhex-4-enyl)-5,11,20,23-tetraisobutyl-8,26-diisopropyl-1,4,10,14,17,19,22,25,28-nonamethyl-3,6,9,12,15,18,21,24,27,30,33-undecaoxo-1,4,7,10,13,16,19,22,25,28,31-undecaazacyclotritriacontan-2-yl)methyl)benzoate.

This was used in the next reaction without further purification.

To a stirred solution of the yellow solid residue (0.93 g) in THF:MeOH (1:1, 20 ml) at 0° C. was added dropwise a solution of LiOH.H2O (500 mg) in water (10 ml). The reaction was allowed to gradually warm-up to room temperature over 18 hours. Then CH₂Cl₂ (20 ml) was added. The resultant solution was acidified with 2M HCl(aq) (pH=3). The separated aqueous layer was extracted with CH₂Cl₂ (3×30 ml). The combined organic extracts were washed with saturated 2M HCl (aq) (2×30 ml) and brine (2×30 ml) and then dried (MgSO₄(s)).

The volatiles were removed al vacuo to leave a solid residue as a mixture of the acid (Intermediate 1) and unreacted cyclosporine A. The acid was separated from the cyclosporine by flash column chromatography through an amine column eluting with a mixture of MeOH:CH₂Cl₂:NH₃(aq) (1:8:1) to give the acid as a salt. After stirring the salt in CH₂Cl₂ (20 ml) and 2M HCl (aq) (20 ml) for 10 minutes, extraction by CH₂Cl₂ (3×20 ml), concentration, and purification by flash column chromatography gave Intermediate 1 (0.300 g, 0.24 mmol, 27%) as a yellow solid.

FAB+ve; Calc. m/z C₇₀H₁₁₇N₁₁O₁₄ (M+Na) 1358.86787. Found (M+Na) 1358.86447.

### Intermediate 2

N-(6-aminohexyl)-4-(((5S,8S,11S,14S,17R,20S,23S,26S,29S,32S)-32-ethyl-29-((1R,2R,E)-1-hydroxy-2-methylhex-4-enyl)-5,11,20,23-tetraisobutyl-8,26-diisopropyl-1,4,10,14,17,19,22,25,28-nonamethyl-3,6,9,12,15,18,21,24,27,30,33-undecaoxo-1,4,7,10,13,16,19,22,25,28,31-undecaazacyclotritriacontan-2-yl)methyl)benzamide.

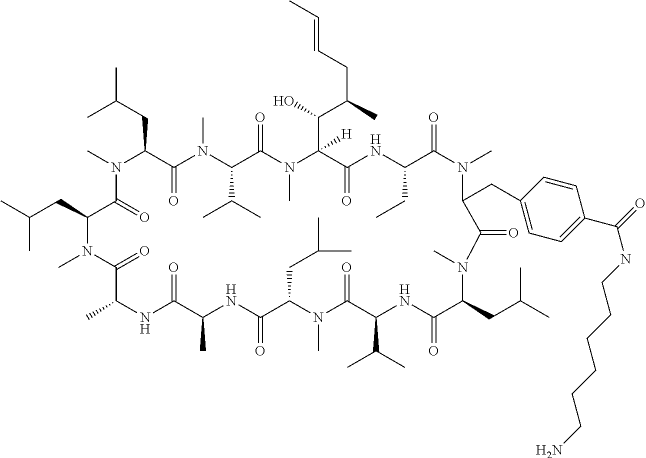

To a stirred solution of Intermediate 1 (109 mg, 0.08 mmol) in dry THF (3.0 ml) was added N-Fmoc-1,6-diaminohexane hydrobromide (68.5 mg, 0.16 mmol), PyBOP (84.5 mg, 0.16 mmol) and triethylamine (0.25 mmol, 0.4 ml) under nitrogen at room temperature and the resultant mixture was stirred for 24 hours. Then CH₂Cl₂ (5 ml) followed by saturated aqueous ammonium chloride (5 ml) were added. The mixture was extracted with CH₂Cl₂ (2×3 ml), dried (MgSO₄(s)).

The volatiles were removed al vacuo to leave a brown oil residue. Purification by chromatography gave the Fmoc-protected derivative (100 mg): (9H-fluoren-9-yl)methyl 6-(4-(((5S,8S,11S,14S,17R,20S,23S,26S,29S,32S)-32-ethyl-29-((1R,2R,E)-1-hydroxy-2-methylhex-4-enyl)-5,11,20,23-tetraisobutyl-8,26-diisopropyl-1,4,10,14,17,19,22,25,28-nonamethyl-3,6,9,12,15,18,21,24,27,30,33-undecaoxo-1,4,7,10,13,16,19,22,25,28,31-undecaazacyclotritriacontan-2-yl)methyl)benzamido)hexylcarbamate as a yellow solid.

This was used without further purification.

A solution of the Fmoc-protected derivative (100 mg) was stirred in 20% piperidine in DMF (4 ml) under argon for 24 hours. The volatiles were removed al vacuo to leave a yellow oil. The oil was purified by flash column chromatography on silica gel eluting with 6% MeOH in DCM followed by MeOH:DCM:NH₃(aq) (1:8:1) to afford the title compound Intermediate 2 (70 mg, 0.05 mmol, 85%) as a yellow solid.

MSES+ve; m/z C₇₆H₁₃₁N₁₃O₁₃ (M+1) 1435.00, (M+2) 718. Found: (M+1) 1435.53, (M+2) 718.76

### Compound 1

(6-(6-(4-(((5S,8S,11S,14S,17R,20S,23S,26S,29S,32S)-32-ethyl-29-((1R,2R,E)-1-hydroxy-2-methylhex-4-enyl)-5,11,20,23-tetraisobutyl-8,26-diisopropyl-1,4,10,14,17,19,22,25,28-nonamethyl-3,6,9,12,15,18,21,24,27,30,33-undecaoxo-1,4,7,10,13,16,19,22,25,28,31-undecaazacyclotritriacontan-2-yl)methyl)benzamido)hexylamino)-6-oxohexyl)triphenylphosphonium

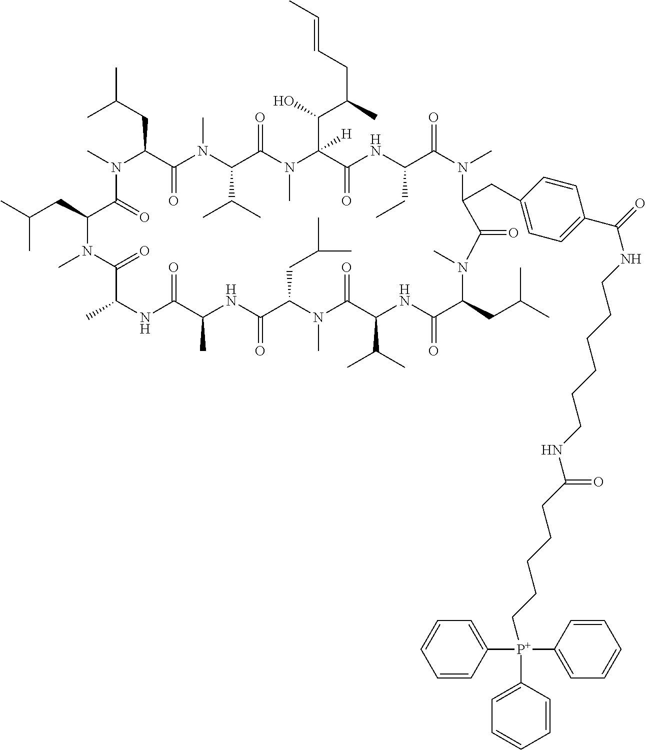

To a stirred solution of the amine Intermediate 2 (65 mg, 0.05 mmol) in dry THF (3 ml) under argon at room temperature was added in one portion PyBOP (35.5 mg, 0.07 mmol), 5-(carboxypentyl)triphenylphosphonium bromide (32 mg, 0.07 mmol) and triethylamine (0.15 mmol, 0.05 ml) and the resultant mixture stirred for 24 hours at room temperature.

The volatiles were removed al vacuo to leave a yellow oil. The oil was purified by flash column chromatography on silica gel eluting with 6% MeOH in DCM followed by MeOH:DCM:NH₃(aq) (1:8:1) to afford the title compound Example 1 (55 mg, 0.03 mmol, 70%) as a white solid.

ES+; Calc. m/z C₁₀₀H₁₅₅N₁₃O₁₄P+ (M+1) 1793.8199. Found: (M+1) 1794.8270, (M+2) 898.

### Synthesis of Compound 2

Compound 2 was prepared via Intermediates 3, 4 and 5.

### Intermediate 3

(5R,6R,E)-6-((2S,5S,11S,14S,17S,20S,23R,26S,29S,32S)-5-ethyl-11,17,26,29-tetraisobutyl-14,32-diisopropyl-1,7,10,16,20,23,25,28,31-nonamethyl-3,6,9,12,15,18,21,24,27,30,33-undecaoxo-1,4,7,10,13,16,19,22,25,28,31-undecaazacyclotritriacontan-2-yl)-6-hydroxy-5-methylhex-2-enoic acid

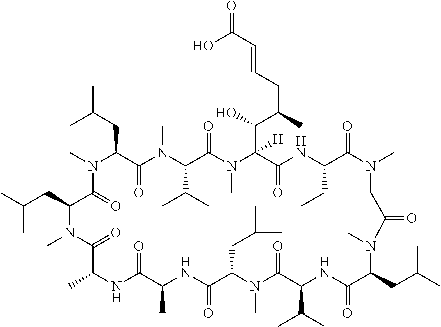

A solution of cyclosporine A (3.70 g, 3.10 mmol), tert-Butyl acrylate (6.36 g, 49.6 mmol, 7.2 ml) and Hoveyda-Grubbs 2^{nd} generation catalyst (155 mg, 0.25, 8%) in dichloromethane (8 ml) was stirred under reflux under argon for 48 hours. The reaction mixture was filtered through celite and the volatiles removed al vacuo to leave a brown oily residue. Purification by flash column chromatography eluting with 6% MeOH in CH₂Cl₂ gave a pale yellow solid (4.00 g), a mixture of the tert-Butyl ester derivative and the unreacted cyclosporine. The solid residue was taken up in a mixture of Trifluoroacetic acid and dichloromethane (10 ml, 1:1) and the mixture stirred for 2 hours at room temperature.

The volatiles were removed al vacuo. The acid was separated from the cyclosporine by flashing the oily residue through a pre-packed amine column eluting with 6% MeOH in DCM followed by MeOH:NH₃(aq):CH₂Cl₂(1:8:1). The acid is eluted as an anion. Acidification of the anion and further purification by flash column chromatography on silica gel eluting with 6% MeOH in DCM afforded Intermediate 3 (1.20 g, 0.93 mmol, 30%) over two steps.

FAB+ve; Calc. m/z C₆₂H₁₀₉N₁₁O₁₄ (M+Na+H) 1255. Found: (M+Na+H) 1255

### Intermediate 4

(9H-fluoren-9-yl)methyl 2-(2-((5R,6R,E)-6-((2S,5S,11S,14S,17S,20S,23R,26S,29S,32S)-5-ethyl-11,17,26,29-tetraisobutyl-14,32-diisopropyl-1,7,10,16,20,23,25,28,31-nonamethyl-3,6,9,12,15,18,21,24,27,30,33-undecaoxo-1,4,7,10,13,16,19,22,25,28,31-undecaazacyclotritriacontan-2-yl)-6-hydroxy-5-methylhex-2-enamido)ethoxy)ethylcarbamate

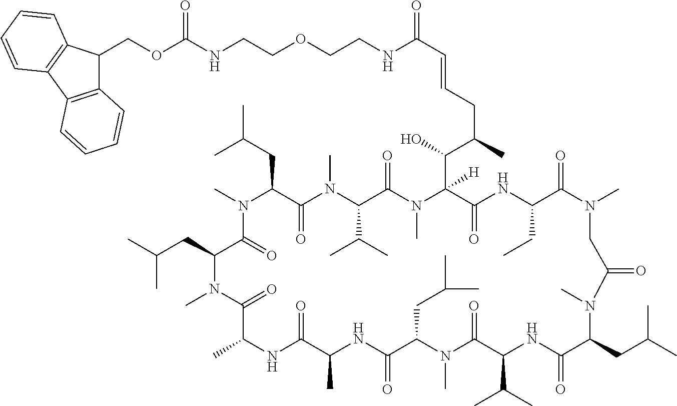

To a solution of Intermediate 3 (560 mg, 0.46 mmol) in dry THF (10 ml) under argon at room temperature was added dropwise, 2-[2-(Fmoc-amino)ethoxyamine]hydrochloride (335 mg, 0.92 mmol), HATU (350 mg, 0.92 mmol) and triethylamine (1.50 mmol, 0.21 ml). The mixture was stirred at room temperature for 24 hours. The volatiles were then removed al vacuo.

The remaining oily residue was purified by flash column chromatography eluting with 6% MeOH in dichloromethane to afford the title compound Intermediate 4 (638.00 g, 0.42 mmol, 90%) as a white solid.

TOF MS ES+; Calc. m/z C₈₁H₁₂₉N₁₃O₁₆ (M+Na) 1562.9578. Found: (M+Na) 1562.9580.

### Intermediate 5

(5R,6R,E)-N-(2-(2-aminoethoxy)ethyl)-6-(2S,5S,11S,14S,17S,20S,23R,26S,29S,32S)-5-ethyl-11,17,26,29-tetraisobutyl-14,32-diisopropyl-1,7,10,16,20,23,25,28,31-nonamethyl-3,6,9,12,15,18,21,24,27,30,33-undecaoxo-1,4,7,10,13,16,19,22,25,28,31-undecaazacyclotritriacontan-2-yl)-6-hydroxy-5-methylhex-2-enamide.

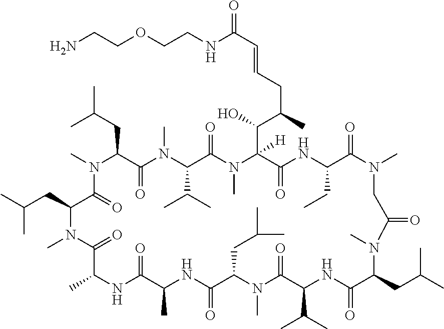

A solution of Intermediate 4 (500 mg, 0.33 mmol) in a mixture of 20% piperidine in DMF (5 ml) was stirred for at room temperature for 3 hours. The volatiles were removed al vacuo to leave a yellow oily residue which was purified by flash column chromatography eluting with 6% MeOH in dichloromethane followed by MeOH:NH₃(aq):CH₂Cl₂(1:8:1) to afford the title compound Intermediate 5 (382 mg, 0.29 mmol, 90%) as a white solid.

FAB+ve; Calc. m/z C₆₆H₁₁₉N₁₃O₁₄(M+Na) 1340.88967. Found: (M+Na) 1340.89380.

### Compound 2

(16R,17R,E)-17-((2S,5S,11S,14S,17S,20S,23R,26S,29S,32S)-5-ethyl-11,17,26,29-tetraisobutyl-14,32-diisopropyl-1,7,10,16,20,23,25,28,31-nonamethyl-3,6,9,12,15,18,21,24,27,30,33-undecaoxo-1,4,7,10,13,16,19,22,25,28,31-undecaazacyclotritriacontan-2-yl)-17-hydroxy-16-methyl-4,12-dioxo-1,1,1-triphenyl-8-oxa-5,11-diaza-1-phosphoniaheptadec-13-ene

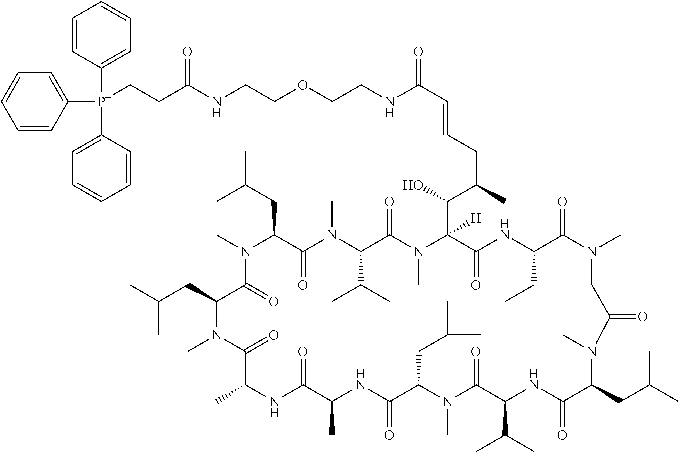

To a solution of Intermediate 5 (288 mg, 0.22 mmol) in THF (5 ml) was added (2-Carboxyethyl)triphenylphosphonium bromide (182 mg, 0.44 mmol), HATU (166 mg, 0.44 mmol) and triethylamine (0.70 mmol, 0.1 ml) under argon at room temperature. The reaction mixture was stirred for 24 hours at room temperature.

The volatiles were removed al vacuo to leave a yellow oily residue which was purified by flash column chromatography eluting with 6% MeOH in dichloromethane followed by MeOH:NH₃(aq):CH₂Cl₂ (1:8:1) to afford the title compound Compound 2.

TOF MS ES+; Calc. m/z C₈₇H₁₃₇N₁₃O₁₅P+(M+1) 1635.0095. Found: (M+1) 1636.0115

### Synthesis of Compound 3

Compound 3 was prepared from Intermediate 1 via Intermediate 6.

### Intermediate 6

N-(2-(2-aminoethoxy)ethyl)-4-(5S,8S,11S,14S,17R,20S,23S,26S,29S,32S)-32-ethyl-29-((1R,2R,E)-1-hydroxy-2-methylhex-4-enyl)-5,11,20,23-tetraisobutyl-8,26-diisopropyl-1,4,10,14,17,19,22,25,28-nonamethyl-3,6,9,12,15,18,21,24,27,30,33-undecaoxo-1,4,7,10,13,16,19,22,25,28,31-undecaazacyclotritriacontan-2-yl)methyl)benzamide

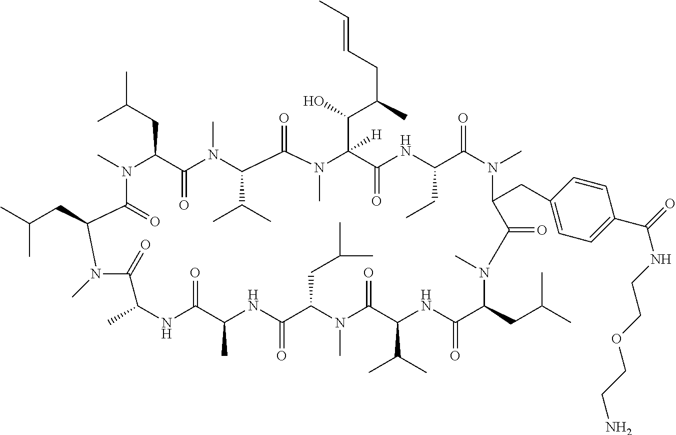

To a stirred solution of the Intermediate 1 (100 mg, 0.07 mmol) in dry THF (6.0 ml) was added 2-[2-(Fmoc-amino)ethoxy]amine hydrochloride (70.0 mg, 0.12 mmol), HATU (70.0 mg, 0.12 mmol) and triethylamine (0.36 mmol, 0.1 ml) under nitrogen at room temperature and the resultant mixture was stirred for 24 hours. Then CH₂Cl₂ (5 ml) followed by saturated aqueous ammonium chloride (5 ml) were added. The mixture was extracted with CH₂Cl₂ (2×3 ml), dried (MgSO₄(s)).

The volatiles were removed al vacuo to leave a brown oil residue. Purification by chromatography gave the Fmoc-protected derivative (100 mg, 0.61 mmol, 82%): (9H-fluoren-9-yl)methyl 2-(2-(4-(((5S,8S,11S,14S,17R,20S,23S,26S,29S,32S)-32-ethyl-29-((1R,2R,E)-1-hydroxy-2-methylhex-4-enyl)-5,11,20,23-tetraisobutyl-8,26-diisopropyl-1,4,10,14,17,19,22,25,28-nonamethyl-3,6,9,12,15,18,21,24,27,30,33-undecaoxo-1,4,7,10,13,16,19,22,25,28,31-undecaazacyclotritriacontan-2-yl)methyl)benzamido)ethoxy)ethylcarbamate as a white solid.

This was used without further purification.

A solution of the Fmoc protected derivative (90 mg) was stirred in 20% piperidine in DMF (4 ml) under argon for 24 hours. The volatiles were removed al vacuo to leave a yellow oil. The oil was purified by flash column chromatography on silica gel eluting with 6% MeOH in DCM followed by MeOH:DCM:NH₃(aq) (1:8:1) to afford the title compound Intermediate 6: (55 mg, 0.04 mmol, 80%) as a yellow solid.

MSES+ve, m/z 1424.47 (M+1), 712.24 (M+2)

### Compound 3

12-(4-(((5S,8S,11S,14S,17R,20S,23S,26S,29S,32S)-32-ethyl-29-((1R,2R,E)-1-hydroxy-2-methylhex-4-enyl)-5,11,20,23-tetraisobutyl-8,26-diisopropyl-1,4,10,14,17,19,22,25,28-nonamethyl-3,6,9,12,15,18,21,24,27,30,33-undecaoxo-1,4,7,10,13,16,19,22,25,28,31-undecaazacyclotritriacontan-2-yl)methyl)phenyl)-4,12-dioxo-1,1,1-triphenyl-8-oxa-5,11-diaza-1-phosphoniadodecane

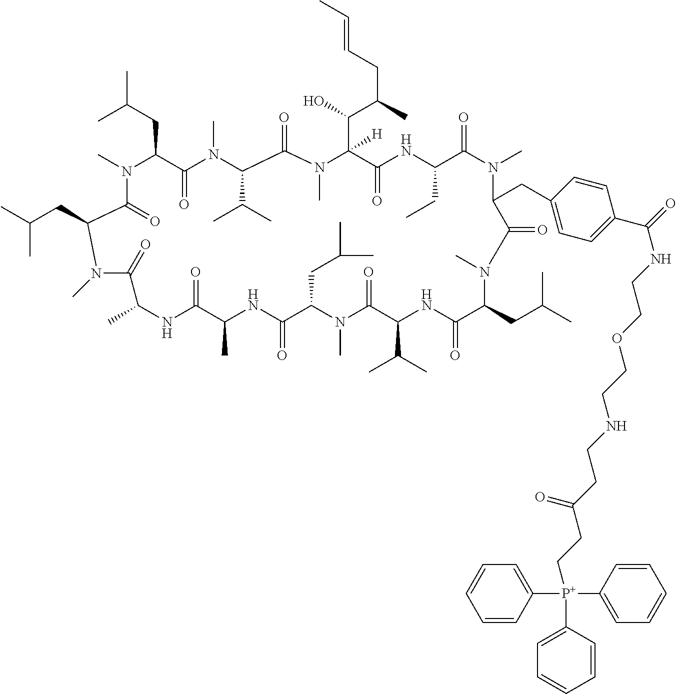

To a solution Intermediate 6 (50 mg, 0.035 mmol) in THF (1 ml) was added (2-Carboxyethyl)triphenylphosphonium bromide (30 mg, 0.07 mmol), HATU (30 mg, 0.07 mmol) and triethylamine (0.70 mmol, 0.1 ml) under argon at room temperature. The reaction mixture was stirred for 24 hours at room temperature. The volatiles were removed al vacuo to leave a yellow oil. The oil was purified by flash column chromatography on silica gel eluting with 6% MeOH in DCM followed by MeOH:DCM:NH₃(aq) (1:8:1) to afford Compound 3 (40 mg, 0.029 mmol, 82%) as a white solid.

MSES+ve; m/z C₉₅H₁₄₅N₁₃O₁₅P+ 1424.47. Found: 1424.47

### Compound 4

Compound 4 depicted below was prepared from Intermediate 2 over a number of steps analogous to those described above.

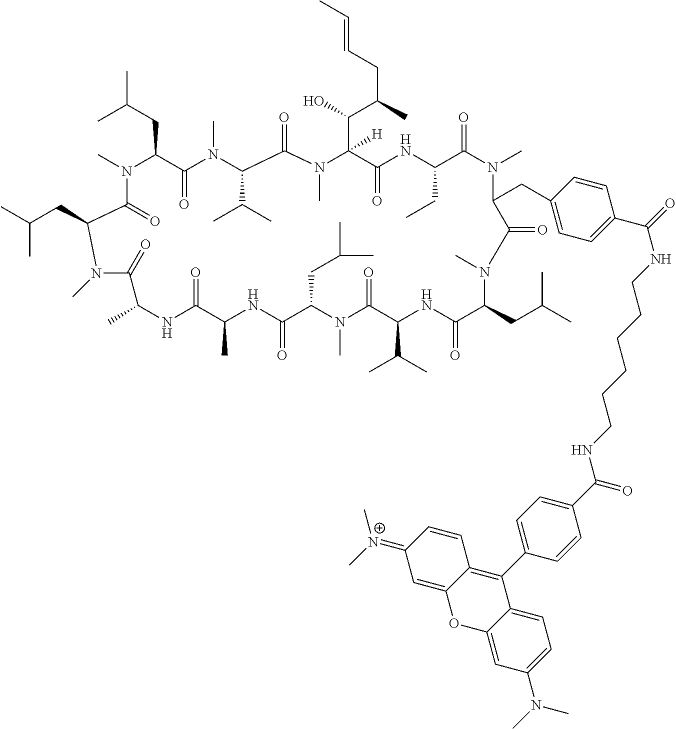

### Synthesis of Compound 5

Compound 5 was prepared via Intermediates 7, 8 and 9.

### Intermediate 7

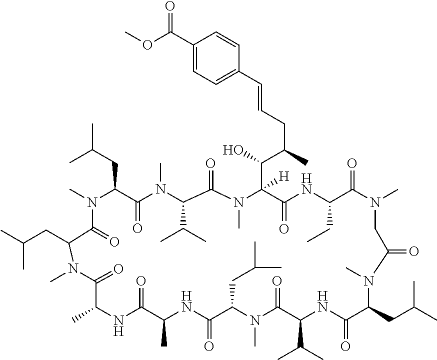

A solution of cyclosporine A (1.00 g, 0.832 mmol), methyl-4-vinylbenzoate (270 mg, 1.665 mmol) and Hoveyda-Grubbs 2^{nd} generation catalyst (20 mg, 0.032, 4%) in dichloromethane (4 ml) was stirred at reflux (60° C.) under nitrogen for 48 hours. TLC analysis (acetone:cyclohexane, 1:1) of the reaction mixture showed the presence of the product (R_{f} 0.63) and complete consumption of the cyclosporine A starting material (R_{f} 0.65). LCMS analysis also confirmed the presence of the product.

The reaction mixture was pre-absorbed on silica gel and purified by flash column chromatography (ethyl acetate:cyclohexane, 1:1 to ethyl acetate to ethyl acetate:methanol, 10%) and the solvent removed in vacuo to give a grey solid. The grey solid was then further purified by removing the Grubbs-Hoveida catalyst by letting it through an SPE-thiol column (eluant: methanol).

The solvent was removed in vacuo to give the Intermediate 7 as a white crystalline solid (950 mg, 86.4%).

HRMS (TOF MS ES⁺): found 1344.8726 [M+Na]⁺ C₆₉H₁₁₅N₁₁O₁₄Na requires 1344.8523; Calculated isotopic distribution: 1344.8523 (100%), 1345.8553 (82.9%), 1346.8584 (32.5%), 1347.8612 (11.4%); Found isotopic distribution: 1344.8726 (100%), 1345.8918 (89.2%), 1346.9116 (35.7%), 1347.9224 (7.1%); vₘₐₓ (thin film, KBr): 3466, 3418, 3318 (m-s, bumpy, CON-Hs, OH), 2961, 2935, 2873 (m, alkyl C—H), 1720 (m, conjugated C═OOMe), 1627 (s broad, bumpy, C═Os, amide I), 1520 (m broad, bumpy, C═Os, amide II) cm⁻¹; δ_{H} (CDCl₃, 500 MHz): 7.92 (1H, d, J 10.5 Hz, NH), 7.90 (2H, d, J_{ArCH,ArCH} 8.4 Hz, 2×ArHs), 7.64 (1H, d, J 7.6 Hz, NH), 7.47 (1H, d, J 8.2 Hz, NH), 7.32 (2H, d, J_{ArCH,ArCH} 8.4 Hz, 2×ArHs), 7.06 (1H, d, J 7.9 Hz, NH), 6.31-6.24 (2H, m, H^{A} & H^{B}), 3.17 (3H, s, OMe methyl ester), 2.65-2.60 (1H, m, completely hidden under other peaks, H^{C2}), 1.88-1.79 (1H, m, partly hidden, H^{C1}); δ_{C} (CDCl₃, 125 MHz): 173.84 (C═O), 173.81 (C═O), 173.74 (C═O), 173.50 (C═O), 171.54 (C═O), 171.31 (C═O), 171.17 (C═O), 170.50 (C═O), 170.44 (2×C═O), 170.26 (C═O), 170.18 (C═O), 167.1 (C═OOMe), 142.4 (Cq-C^{A}), 132.9 (C^{A}), 130.6 (C^{B}), 129.9, 125.9 (4Cs, 4×ArCs), 128.2 (Cq-COOMe), 52.0 (OMe methyl ester), 36.8 (C^{C}).

### Intermediate 8

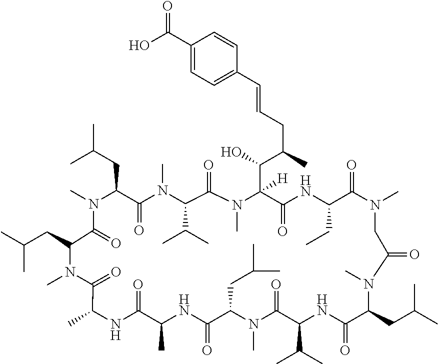

Intermediate 7 (260 mg, 0.196 mMol) was stirred in acetone (4 mL) and an aqueous solution of sodium hydroxide (2M, 2 mL). After 19 hours a white precipitate had formed and T.1.c. analysis (acetone:cyclohexane, 1:1) showed the presence of one product (R_{f} 0.17) and some residual starting material/impurity (R_{f} 0.31). The acetone was removed from the reaction mixture and the aqueous layer left behind was washed with ethyl acetate. The aqueous layer was acidified with an aqueous solution of hydrochloric acid (1M) and washed again with ethyl acetate. The collected ethyl acetate layers were dried (magnesium sulfate), filtered and concentrated in vacuo to give a white/pale brown hygroscopic solid which was then diluted in acetonitrile and filtered again (eluant acetonitrile). The filtrate was finally concentrated in vacuo to give Intermediate 8 (220 mg, 86%) as a white/pale brown hygroscopic solid. vₘₐₓ (thin film, KBr): 3418, 3315 (m, bumpy, CON-Hs, OH), 2961, 2936, 2873 (m, alkyl C—H), 1714 (m, conjugated C═OOH), 1627 (s broad, bumpy, C═Os, amide I), 1520 (m broad, bumpy, C═Os, amide II) cm⁻¹

### Intermediate 9

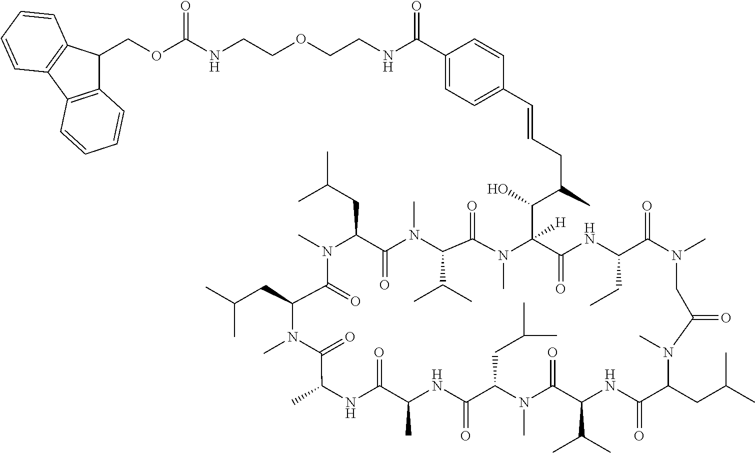

HATU coupling reagent (230 mg, 0.6037 mMol) was added to a solution of Intermediate 8 (395 mg, 0.3018 mMol), chloroform (10 mL) and triethylamine (168 μL) which had been stirring for 5 minutes under an atmosphere of nitrogen at room temperature. After a further 5 minutes 2-[2-(Fmoc-amino)ethoxy ethylamine hydrochloride (257 mg, 0.7083 mMol) was added to the stirring reaction mixture and left to react for 22.5 hours. LCMS analysis revealed the presence of the product in the reaction mixture. The reaction mixture was concentrated in vacuo and successively diluted in ethyl acetate and washed with an aq hydrochloric acid solution (1M). The collected organic layers were dried over magnesium sulphate, filtered and concentrated in vacuo to give a residue which was purified by flash column chromatography (chloroform to chloroform:methanol, 3%) to give Intermediate 9 (406 mg, 83%) as a white hygroscopic solid.

### Compound 5

Compound 5 was prepared from Intermediate 9 using techniques analogous to those described above.

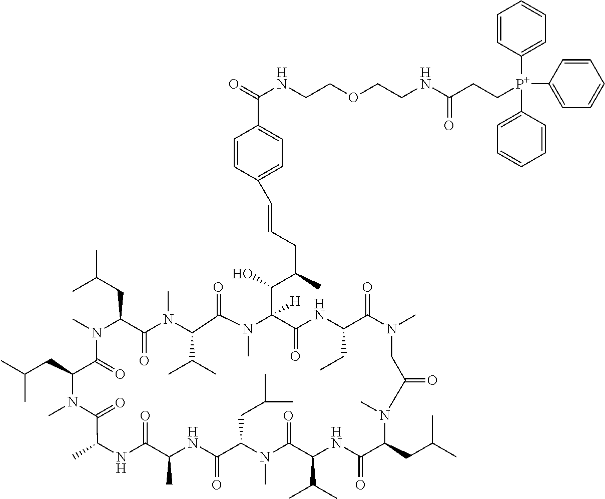

### Compound 6

Compound 6 depicted below was prepared over a number of steps analogous to those described above.

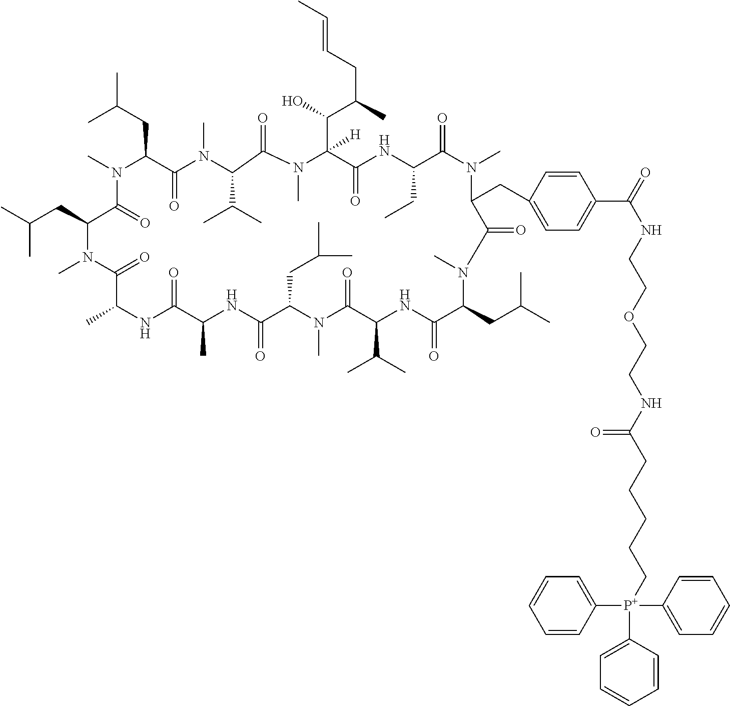

### Compound 7

Compound 7 depicted below was prepared over a number of steps analogous to those described above.

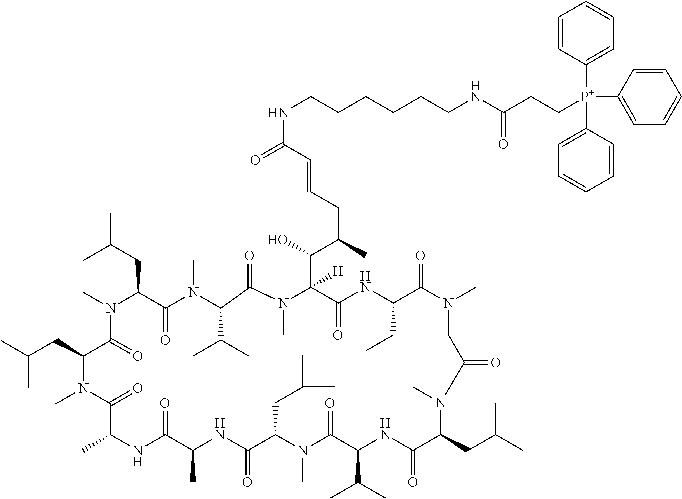

### Analysis of the Properties of Compound 1

### Example 1

### Interactions of Compound 1 with Cyclophilins and Calcineurin

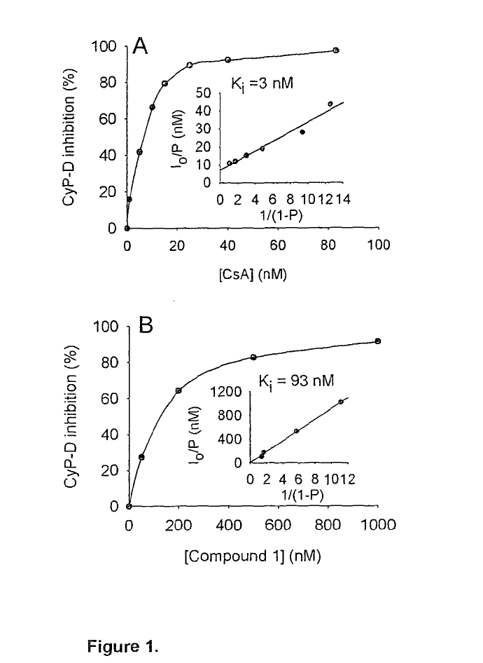

Cyclophilins are peptidylprolyl cis-trans isomerases; this activity is inhibited by cyclosporins. Compound 1 and CsA interactions with cyclophilin D (CyP-D) and cyclophilin A (CyP-A) were investigated from the inhibition of peptidylprolyl cis-trans-isomerase (PPIase) activity. About 7 nM (total) CsA yielded 50% inhibition of CyP-D (see FIG. 1A). However, this underestimates the true CsA binding affinity since assays contained a similar concentration of CyP-D (8 nM). Accordingly, inhibition was analysed using the Henderson equation for a tight-binding inhibitor (see above), which gave an inhibitory (dissociation) constant for CsA and CyP-D of 3 nM (inset FIG. 1A).

Analogous analyses for Compound 1 (FIG. 1B) and Intermediates 1 and 2 showed that increasing addition to position 3 increasingly impaired binding, so that the binding affinity of Compound 1 to CyP-D was about 30-fold lower than CsA. The binding affinities of CsA and Compound 1 to CyP-A were similar to those with CyP-D. These figures are shown in Table 1 below.

TABLE 1

| Compound | Kᵢ for CyP-D (nM) | Kᵢ for CyP-A (nM) |
|---|---|---|
| Cyclosporin A | 3 | 4 |
| Compound 1 | 93 | 113 |

In addition to inhibiting cyclophilins, CsA forms a complex with CyP-A that, in turn, inhibits the Ca²⁺/calmodulin-dependent Ser/Threo protein phosphatase calcineurin thereby enlarging considerably its sphere of action. It was important, therefore, to establish how the 3-position modification affected the capacity of the complex to inhibit calcineurin.

To enable comparisons, the concentrations of CsA (1 μM) and Compound 1 (4 μM) in the test incubations were chosen to establish the same concentrations of the CsA/CyP-A and Compound 1/CyP-A complexes ie. 720 nM complex with 740 nM total CyP-A (calculated using the Kᵢ values of CyP-A with CsA and Compound 1).

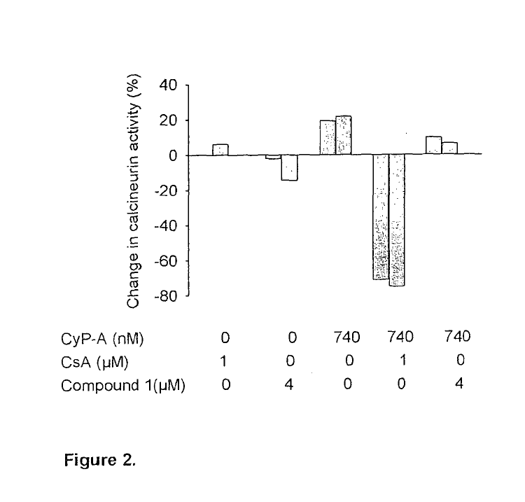

FIG. 2 shows that, whereas CyP-A alone produced a small (20%) activation of calcineurin, the CsA/CyP-A complex (720 nM) inhibited by about 70%. In contrast, the Compound 1/CyP-A complex (720 nM) produced no inhibition. Conjugation to position 3 of the CsA ring appears to prevent formation of the ternary cyclophilin /cyclosporin/calcineurin complex which is known to require interactions between calcineurin and positions 3-7 of the ring.

### Example 2

### Evaluation of the CyP-D Selectivity of Compound 1 in a Mixed In Vitro System

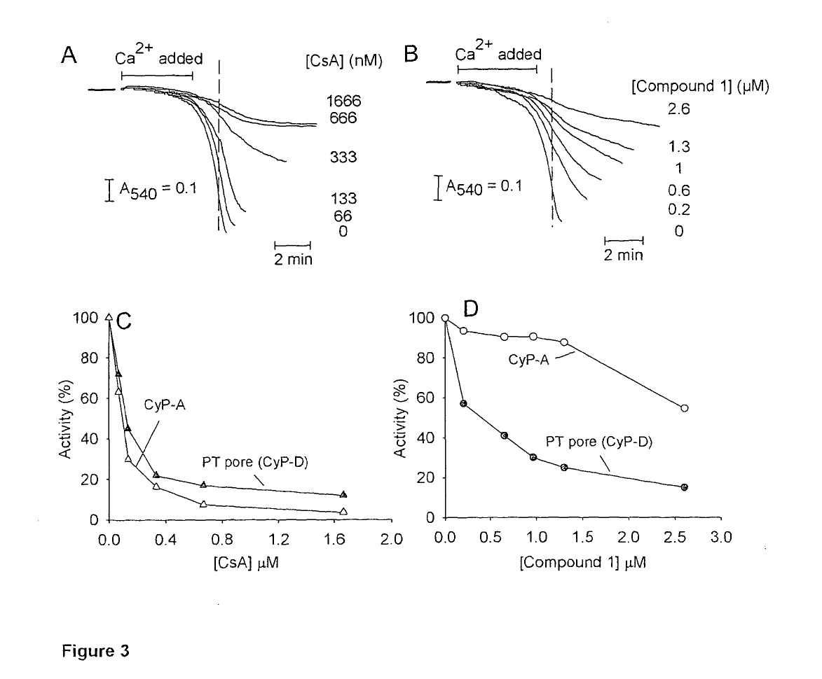

It was investigated whether Compound 1 would select for intramitochondrial CyP-D and the PT pore, rather than extramitochondrial cyclophilins, using a test system comprising isolated mitochondria and externally-added, recombinant CyP-A. To evaluate CyP-D activity in mitochondria, formation of the Permeability Transition (PT) pore in mitochondria was monitored. PT pore formation is controlled by CyP-D, such that CyP-D inhibition prevents PT pore formation. PT pore opening was induced by addition of high [Ca²⁺], and was monitored by the resultant mitochondrial swelling as the inner membrane became freely permeable to low Mr solutes. Swelling was monitored by the decrease in absorbance at 540 nm (FIGS. 3A and 3B).

As Ca²⁺ influx into mitochondria is electrophoretic, the inner membrane potential, Δφ_{M}, becomes dissipated during rapid Ca²⁺ uptake (and then restored when uptake is complete). Since dissipation of Δφ_{M} would compromise accumulation of the positively-charged Compound 1 in mitochondria, Ca²⁺ was infused slowly into the test incubations to limit the rate of Ca²⁺ uptake and thereby avoid membrane depolarisation (this was confirmed using a tetraphenylphosphonium electrode and CsA to block PT pore opening).

CsA and Compound 1 inhibited pore opening as shown in FIGS. 3A and 3B, indicating inhibition of CyP-D. Estimation of the degrees of inhibition (from the decreases in absorbance attained at the time marked by the dashed lines), indicate that about 0.1 μM CsA and 0.4 μM Compound 1 gave 50% inhibition of pore opening (closed symbols; FIGS. 3C and 3D).

Samples were also withdrawn immediately after Ca²⁺ addition, the mitochondria sedimented, and CyP-A activity in the supernatant determined (open symbols).

From these figures it can be seen that CsA inhibited extramitochondrial CyP-A with a concentration profile similar to that of the PT (FIG. 3C); this was expected since CyP-D and CyP-A have similar binding affinities for CsA and, being uncharged, CsA should equilibrate to the same free concentrations on either side of the inner membrane.

In contrast, Compound 1 inhibited PT pore formation considerably better than it inhibited CyP-A (FIG. 3D), even though it binds to CyP-A and CyP-D with similar affinities (Table 1). This indicates that Compound 1 was accumulated in the mitochondrial matrix (where CyP-D is located) with respect to the external medium (containing CyP-A).

From these data, an effective mitochondrial matrix/extramitochondrial accumulation ratio due to mitochondria targeting can be calculated. The mitochondrial matrix/extramitochondrial accumulation ratio is equal to:

$$\frac{50}{\begin{matrix}CyP \text{-} A inhibition ( \% ) at the [ conjugate ] yielding \\ 50 \% inhibition of PT pore\end{matrix}} \cdot \frac{{K}_{i} for CyPD}{{K}_{i} for CyPA}$$

This gives a value of 4.8 for Compound 1 and 0.6 for unmodified CsA.

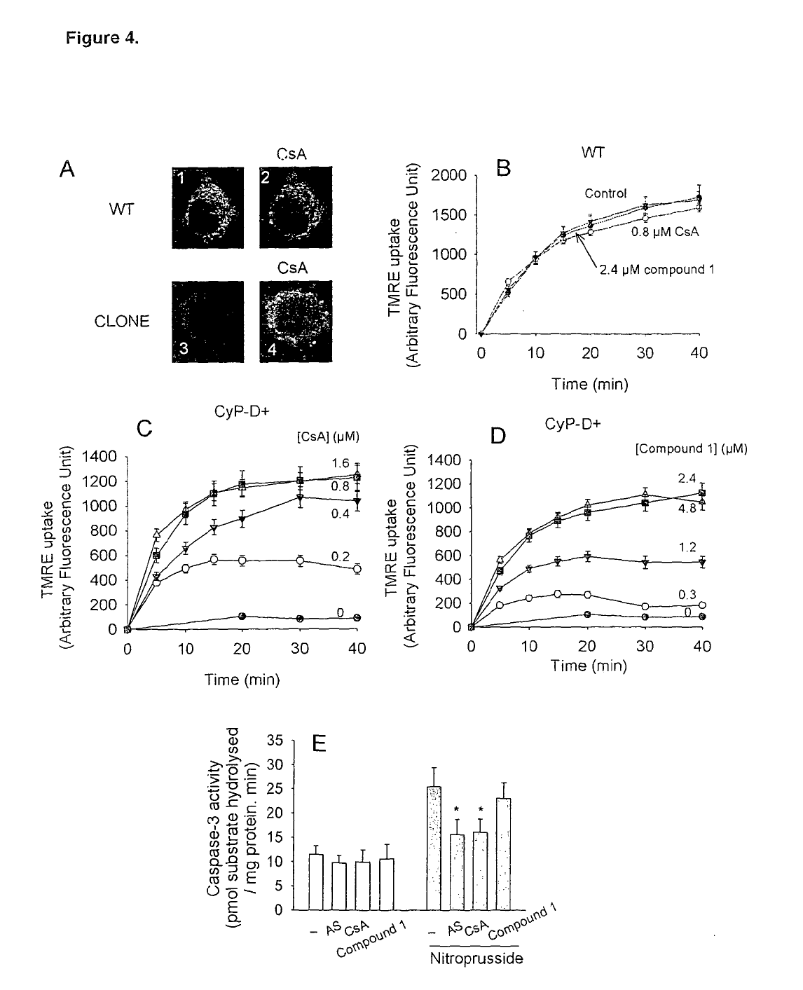

The data of FIG. 4 show that, unlike CsA, Compound 1 preferentially inhibits intramitochondrial CyP-D rather than extramitochondrial CyP-A.

### Example 3

### Evaluation of the CyP-D Selectivity of Compound 1 in Intact Cells

Selectivity of Compound 1 for CyP-D in intact cells was investigated using rat B50 neuroblastoma cells and a clone in which CyP-D is overexpressed about 10-fold {CyP-D(+) cells}. CyP-D(+) cells maintain a relatively low Δφ_{M}, indicative of transient PT pore opening. Since the lowering of Δφ_{M} is caused by excessive CyP-D, restoration of Δφ_{M} to wild-type values provides an unequivocal measure of CyP-D inhibition.

Changes in Δφ_{M} were monitored from the uptake of tetramethylrhodamine ethylester (TMRE), a fluorescent, lipophilic cation accumulated by mitochondria according to the magnitude of the potential.

FIG. 4A shows typical images of TMRE accumulated within the mitochondria of these cells. Mitochondria of the CyP-D (+) clone accumulated considerably less TMRE than wild type cells, but the difference was removed by CsA, which promoted uptake by the CyP-D(+) cells. Maximal restoration of TMRE uptake by CyP-D(+) cells was obtained with about 0.8 μM CsA (FIG. 4C) and 2.4 μM Compound 1 (FIG. 4D). The same concentrations did not affect TMRE uptake by wild type cells (FIG. 4B). It may be concluded that about 0.8 μM CsA and 2.4 μM Compound 1 are sufficient to inhibit CyP-D in B50 cells.

To investigate whether Compound 1 inhibited CyP-D selectively ie. without appreciable inhibition of CyP-A, a marker of CyP-A activity was required. Caspase activation in B50 cells induced by nitroprusside is diminished by CsA and by antisense (AS) suppression of CyP-A , indicating an involvement of CyP-A in caspase activation in this model. This system offers a measure of CyP-A activity.

Antisense treatment decreased CyP-A expression by >85%, and antisense treatment and CsA both reduced nitroprusside-induced activation of caspase-3 (FIG. 4E). Unlike CsA, however, 2.5 μM of Compound 1 had no significant effect on caspase activation. Thus, Compound 1 shows selectivity for mitochondrial CyP-D over cytosolic CyP-A in intact cells.

These data indicate that, unlike CsA, Compound 1 is accumulated by mitochondria from the cytosol.

### Example 4

### Ischaemia/Reperfusion in Hippocampal Neurons

Ischaemia/reperfusion (I/R) was mimicked by incubating hippocampal neurons under oxygen and glucose deprivation (OGD) for 30 min, after which glucose and O₂ were restored. To indicate the time period of OGD needed to remove O₂ sufficiently for impairment of mitochondrial electron transport, TMRE loss from mitochondria of preloaded cells was followed as an index of Δφ_{M} dissipation.

TMRE was lost after about 5 min OGD indicating respiratory inhibition at this time. At the outset of each experiment, a group of hippocampal neurons were distinguished from underlying glial cells and the same neurons were imaged at intervals thereafter. The susceptibility of neuronal cells (but not glial cells) to OGD-induced necrosis increased with days in culture, and data were obtained after culture for 24-28 days.

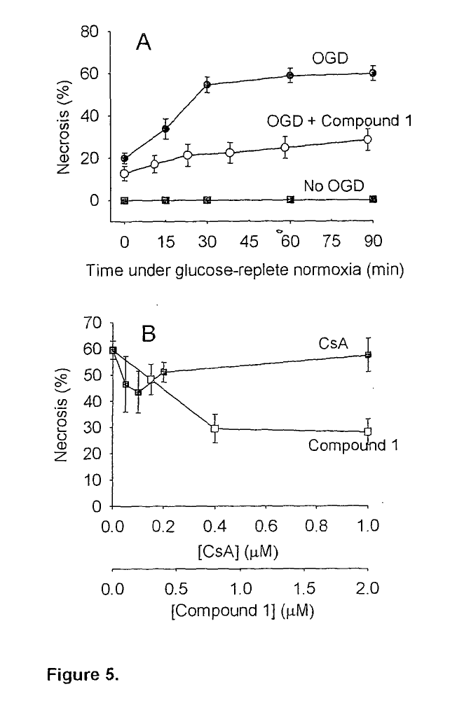

Following OGD, about 60% of neurons became necrotic within 90 min (FIG. 5A), but mortality was approximately halved in the presence of Compound 1. Maximal protection was given with >0.8 μM Compound 1 (FIG. 5B).

CsA was less protective (FIG. 5B). There was a relatively small protection with 0.1 μM CsA 1, but this was reversed at higher CsA concentrations, indicating the existence of secondary CsA targets outside mitochondria that overrode the protection. Thus, restricting the action of CsA to mitochondria, using a cyclosporin conjugated to a mitochondrial targeting group, improves its protective capacity against cell necrosis brought about by a period of OGD, indicating that CyP-D and the PT are major contributors to this form of injury.

### Analysis of the Properties of Compounds 2 to 4

### Example 5

### Interactions with Cyclophilin D

The binding affinities of Compounds 2 to 4 with cyclophilin D were determined as described in Example 1. The results are shown (together with results for CsA and Compound 1) in Table 2 below.

TABLE 2

| Compound | Kᵢ for CyP-D (nM) |
|---|---|
| CsA | 3 |
| Compound 1 | 93 |
| Compound 2 | 30 |
| Compound 3 | 6 |
| Compound 4 | 120 |

### Example 6

### Mitochondrial Matrix/Extramitochondrial Accumulation Ratio

The mitochondrial matrix/extramitochondrial accumulation ratios of Compounds 2 to 4 were determined as described in Example 2. The results are shown (together with results for CsA and Compound 1) in Table 2 below.

TABLE 2

| Compound | Accumulation ratio |
|---|---|
| CsA | 0.6 |
| Compound 1 | 4.8 |
| Compound 2 | >10 |
| Compound 3 | >10 |
| Compound 4 | 4.2 |

### Example 7

### Ischaemia/Reperfusion in Hippocampal Neurons

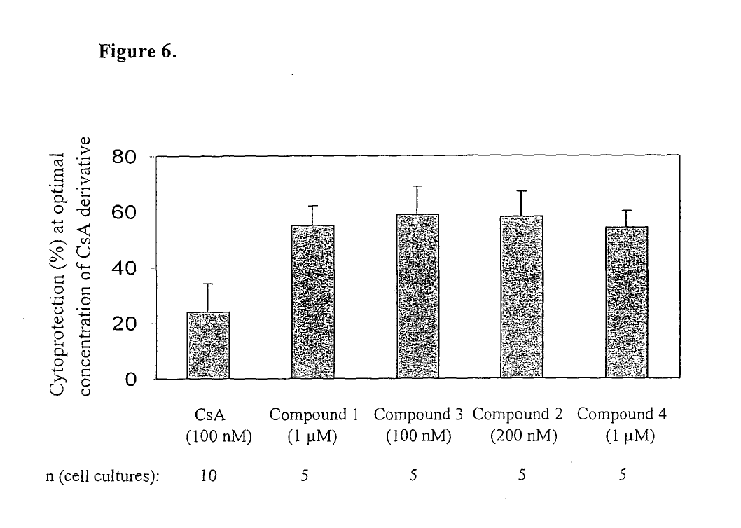

Maximal cytoprotection (%) against ischamia/reperfusion-induced necrosis of rat hippocampal neurons was measured for CsA and Compounds 1 to 4 using the methods described in Example 4. The results are depicted in FIG. 6.

The data in FIG. 6 show that conjugates of the invention containing different mitochondrial targeting groups and with different linkers and different points of attachment to the cyclosporin ring show improved cytoprotection with respect to CsA.

### Analysis of the Cytoprotective Properties of Compounds 2 and 3 in Heart Cells

### Example 8

### Cytoprotective Properties of Compound 2 in Heart Cells

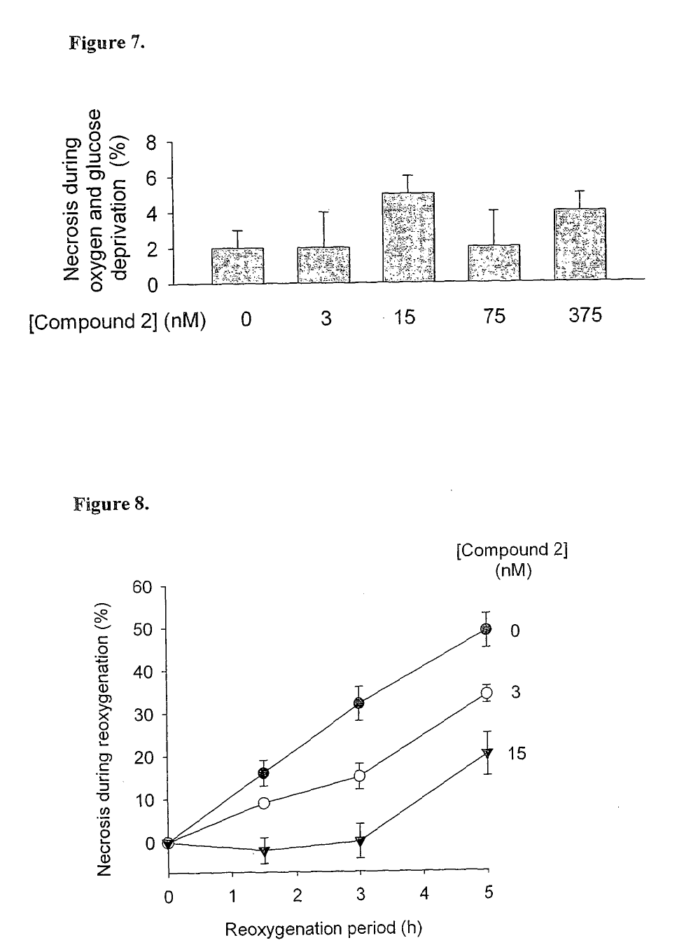

Ischaemia/reperfusion in heart was mimicked by incubating the heart cells under oxygen and glucose deprivation (OGD) for 4 hours, after which oxygen and glucose were restored. As shown in FIG. 7, OGD induced negligible necrosis with or without Compound 2.

However, subsequent reoxygenation in the presence of glucose induced progressive cell death as shown in FIG. 8 below. About 50% of cardiomyocytes became necrotic during 5 hours of reoxygenation. Necrosis during reoxygenation was inhibited by Compound 2, in particular during the first 3 hours of reoxygenation

### Example 9

### Comparison of the Cytoprotective Properties of Compound 2, Compound 3 and CsA in Heart Cells

Cytoprotection against ischaemia/reperfusion injury in heart cells was measured for CsA,

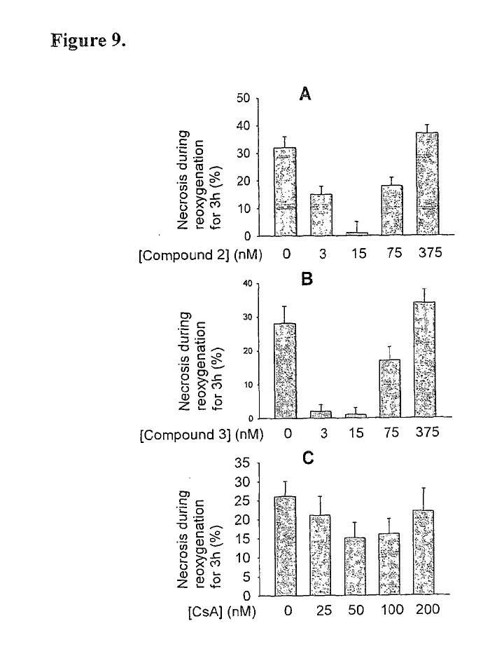

Compound 2 and Compound 3 using the method described in Example 8. Necrosis was determined after 3 hours of reoxygenation. The results are depicted in FIG. 9 below.

The data in FIG. 9 show that both Compound 2 and Compound 3 yield complete protection at a concentration of 15 nM. Compound 3 is also similarly effective at 3 nM. In comparison, the maximal protection by CsA was only 42% and was obtained at a concentration of 50 nM. Thus, Compounds 2 and 3 yield better cytoprotection than CsA and are effective at much lower concentrations than CsA. Mitochondrial targeting markedly improves cytoprotection in heart cells as exemplified by Compounds 2 and 3, which have different linkers and different points of attachment to the cyclosporin ring.

1. A conjugate which comprises a cyclosporin moiety of formula (I) linked to one or more mitochondrial targeting groups, or a pharmaceutically acceptable salt thereof: 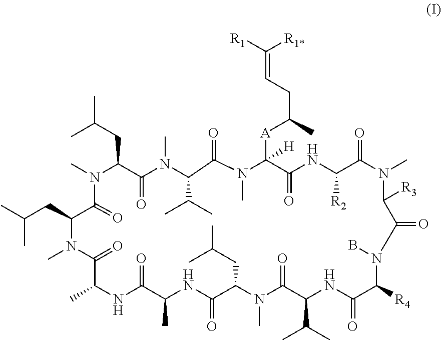
wherein:
A represents 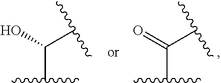
B represents methyl or ethyl, one of R₁ and R₁* represents hydrogen and the other represents methyl,
R₂ represents ethyl or isopropyl,
R₃ represents hydrogen or methyl, and
R₄ represents —CH₂CH(CH₃)CH₃, —CH₂CH(CH₃)CH₂CH₃, —CH(CH₃)CH₃ or —CH(CH₃)CH₂CH₃.

2. A conjugate according to claim 1 or a pharmaceutically acceptable salt thereof, wherein:
A represents 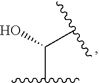
R₁ represents methyl and R₁* represents hydrogen,
B represents methyl,
R₂ represents ethyl,
R₃ represents hydrogen, and
R₄ represents —CH₂CH(CH₃)CH₃.

3. A conjugate according to claim 1 or a pharmaceutically acceptable salt thereof, wherein the mitochondrial targeting group is a lipophilic cation or a mitochondrial targeting peptide.

4. A conjugate according to claim 3 or a pharmaceutically acceptable salt thereof, wherein the lipophilic cation is a phosphonium cation, an arsonium cation, an ammonium cation, flupritine, MKT-077, a pyridinium ceramide, a quinolium, a liposomal cation, a sorbitol guanidine, a cyclic guanidine or a rhodamine.

5. A conjugate according to claim 1 which has the formula (I′) or a pharmaceutically acceptable salt thereof: 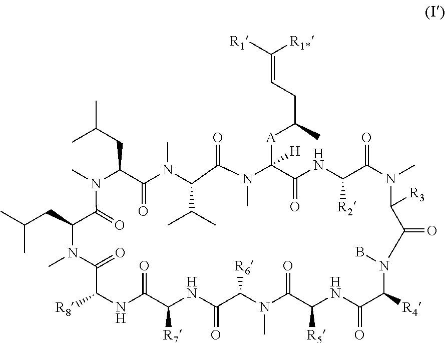
wherein:
one of R₁′ and R₁*′ represents methyl or -L₁-MTG₁ and the other represents hydrogen,
R₂′ represents R₂ as defined in claim 1 or 2 or -L₂-MTG₂,
R₃′ represents R₃ as defined in claim 1 or 2 or -L₃-MTG₃,
R₄′ represents R₄ as defined in claim 1 or 2 or -L₄-MTG₄,
R₅′ represents isopropyl or -L₅-MTG₅,
R₆′ represents —CH₂CH(CH₃)CH₃ or -L₆-MTG₆,
R₇′ represents methyl or -L₇-MTG₇,
R₈′ represents methyl or -L₈-MTG₈, and
A and B are as defined in claim 1 or 2,
L₁ to L₈ independently represents a direct bond or a linker which is a straight chain C_{I} to C₂₀ alkylene which is unsubstituted or substituted by one or more substituents selected from halogen atoms, hydroxy, alkoxy, alkyl, hydroxyalkyl, haloalkyl and haloalkoxy substituents, wherein zero or one to ten carbon atoms in the alkylene chain are replaced by spacer moieties selected from arylene, —O—, —S—, —NR′—, —C(O)NR′— and —C(O)— moieties, wherein R′ is hydrogen or C₁ to C₆ alkyl and the arylene moiety is unsubstituted or substituted by one, two or three substituents selected from halogen atoms, hydroxy, alkyl and alkoxy groups, and
each of MTG₁ to MTG₈ independently represents a mitochondrial targeting group MTG as defined in any one of claims 1, 3 and 4,
provided that at least one and not more than three of R₁′ or R₁*′ and R₂′ to R₈′ represent -L-MTG.

6. A conjugate according to claim 5 wherein R₁′ represents methyl or -L₁-MTG₁, R₁*′ represents hydrogen, R₂′ represents R₂ as defined in claim 1 or 2, R₃′ represents R₃ as defined in claim 1 or 2 or -L₃-MTG₃, R₄′ represents R₄ as defined in claim 1 or 2, R₅′ represents isopropyl, R₆′ represents —CH₂CH(CH₃)CH₃, R₇′ represents methyl, and R₈′ represents methyl.

7. A conjugate according to claim 6 wherein R₁′ represents -L₁-MTG₁ and R₃′ represents hydrogen.

8. A conjugate according to claim 6 wherein R₁′ represents methyl and R₃′ represents -L₃-MTG₃.

9. A conjugate according to claim 7 wherein -L₁-MTG₁ is a compound of formula (VIII): 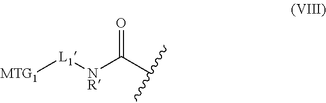
wherein L₁′ represents a straight chain C₁ to C₁₉ alkylene which is unsubstituted or substituted by one or more substituents selected from halogen atoms, hydroxy, alkoxy, alkyl, hydroxyalkyl, haloalkyl and haloalkoxy substituents, wherein 1 to 9 carbon atoms, preferably 1 to 4 carbon atoms, in said alkylene chain are replaced by spacer moieties selected from arylene, —O—, —NR′— and —C(O)NR′— moieties, wherein R′ is hydrogen or C₁ to C₆ alkyl, preferably hydrogen, and the arylene moiety is unsubstituted or substituted by one, two or three substituents selected from halogen atoms, hydroxy, alkyl or alkoxy groups.

10. A conjugate according to claim 8 wherein -L₃-MTG₃ is a compound of formula (IX): 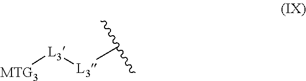
wherein L₃″ represents unsubstituted straight chain C₁ to C₂ alkylene and L₃′ represents C₁ to C₁₈ alkylene which is unsubstituted or substituted by one or more substituents selected from halogen atoms, hydroxy, alkoxy, alkyl, hydroxyalkyl, haloalkyl and haloalkoxy substituents, wherein 1 to 10 carbon atoms, preferably 1 to 4 carbon atoms, in said C₁ to C₁₈ alkylene chain are replaced by spacer moieties selected from arylene, —O—,
—NR′—and —C(O)NR′— moieties, wherein R′ is hydrogen or C₁ to C₆ alkyl, preferably hydrogen, and the arylene moiety is unsubstituted or substituted by one, two or three substituents selected from halogen atoms, hydroxy, alkyl or alkoxy groups.

11. A conjugate according to claim 7 wherein MTG₁ and MTG₃ represent triphenylphosphonium.

12. A conjugate according to claim 7 wherein MTG₁ and MTG₃ represent rosamine.

13. A pharmaceutical composition comprising a conjugate according to claim 1 and a pharmaceutically acceptable excipient, diluent or carrier.

14-17. (canceled)

18. A method of treating a patient suffering from or susceptible to disease or disorder susceptible to amelioration by inhibition of cyclophilin D, which method comprises administering to said patient a conjugate according to claim 1.

19. An experimental assay method employing a conjugate according to claim 1 as a reagent.

20. A method according to claim 14, wherein the disease or disorder is ischaemia/reperfusion injury or neurodegenerative disease.
